(19) United States
(12) Patent Application Publication
(10) Pub. No.: US 2014/0112883 A1
(43) Pub. Date: Apr. 24, 2014

(54) METHODS FOR REDUCING AN ADVERSE IMMUNE RESPONSE TO A FOREIGN ANTIGEN IN A HUMAN SUBJECT WITH ANTI-CD4 ANTIBODIES OR CD4-BINDING FRAGMENTS THEREOF OR CD4-BINDING MOLECULES

(71) Applicants: Paul Ponath, San Francisco, CA (US); Michael Rosenzweig, Boston, MA (US); Lou Vaickus, Hingham, MA (US)

(72) Inventors: Paul Ponath, San Francisco, CA (US); Michael Rosenzweig, Boston, MA (US); Lou Vaickus, Hingham, MA (US)

(73) Assignee: LIQUIDATING TRUST, Rockville, MD (US)

(21) Appl. No.: 14/112,745

(22) PCT Filed: Apr. 13, 2012
(86) PCT No.: PCT/US2012/033483

§ 371 Date: Dec. 11, 2013

Related U.S. Application Data
(60) Provisional application No. 61/477,458, filed on Apr. 20, 2011.

(51) Int. Cl. A61K 39/395; A61K 39/12; A61K 39/00

(52) U.S. Cl. CPC A61K 39/3955; A61K 39/0005; A61K 39/12; A61K 39/0003
USPC 424/85.2; 424/133.1; 424/85.7; 424/85.6; 424/85.1

(57) ABSTRACT

Provided herein are methods of inducing tolerance or reducing an immune response to a foreign antigen in a human subject. The methods include administering anti-CD4 antibodies or CD4-binding fragments thereof or CD4-binding molecules, and the foreign antigen to the human subject.

### TECHNICAL FIELD

This document relates to methods for inducing tolerance or reducing an adverse immune response of a human subject to a foreign antigen, and more particularly to using anti-CD4 antibodies or CD4-binding fragments thereof or CD4-binding molecules, and foreign antigen to induce tolerance or reduce adverse immune response to the foreign antigen.

### BACKGROUND

T helper (Th) cells play a central role in the orchestration of adaptive immunity. B cells require T cell help for affinity maturation and immunoglobulin isotype switching (see, Bishop and Hostager (2001) Curr Opin Immunol., 13(3):278-285; and Mills and Cambier (2003) Semin Immunol., 15(6):325-329). T cell help not only augments expansion of CD8+ T cells but is required for optimal generation of memory CD8+ T cells (see, Janssen, et al. (2003) Nature, 421(6925):852-856; Shedlock and Shen (2003) Science, 300(5617):337-339; and Sun and Bevan (2003) Science, 300(5617):339-342). A key determinant in the differentiation, survival, and effector functions of T cells is the strength of signal received upon encounter with antigen. T cell receptor (TCR) binding to peptide-MHC molecules on an antigen presenting cell (APC) initiates the formation of a signaling complex, the immunological synapse, at the T cell-APC interface. CD4 is a T cell co-receptor that binds directly to non-polymorphic regions of the MHC class II molecules engaged in antigen presentation. Recruitment of CD4 into the immunological synapse amplifies the CD3/TCR signal through provision of lck, a tyrosine kinase non-covalently associated with the cytoplasmic tail of CD4. The development and differentiation of Th cells with specific effector functions is linked to the strength of the signals delivered through the TCR and co-receptors. Therefore, altering or blocking the activity of co-receptors such as CD4 using anti-CD4 antibodies or CD4-binding fragments thereof or molecules can be used to modulate immune responses.

### SUMMARY

This document is based on the use of an anti-CD4 antibody or a CD4-binding fragment thereof or a CD4-binding molecule to mediate antigen-specific hyporesponsiveness to a foreign antigen. As such, anti-CD4 antibodies such as TRX1, or CD4-binding fragments thereof or CD4-binding molecules, can be particularly useful for treating hemophilia and lysosomal storage diseases where neutralizing antibody responses to either factor or enzyme replacement limit therapy and affect patient outcome.

In one aspect, this document features a method of inducing tolerance or reducing an adverse immune response of a human subject to a foreign antigen. The method includes treating the subject with a regimen, where the regimen includes one or more administrations of an anti-CD4 antibody or a CD4-binding fragment thereof or CD4-binding molecule to the subject; and one or more administrations of the foreign antigen to the subject. During a tolerizing window, the total dose of the antibody administered is 30 mg/kg or less, or the total dose of the fragment administered is the biological equivalent of 30 mg/kg or less, and wherein the tolerizing window is 10 days or less. In some embodiments, the subject, prior to the first administration of the antibody, fragment, or molecule, has a detectable level of antibody that binds to the foreign antigen. In some embodiments, the subject, prior to the first administration of the antibody, fragment, or molecule, does not have a detectable level of antibody that binds to the foreign antigen.

In one aspect, this document features a method of inducing tolerance or reducing an immune response of a human subject to a foreign antigen. The method includes treating the subject with a regimen, the regimen includes one or more administrations of an anti-CD4 antibody or a CD4-binding fragment thereof or CD4-binding molecule to the subject, the first administration including at least 0.05 mg/kg but less than 5 mg/kg of the antibody or the biological equivalent of the fragment, and one or more administrations of the foreign antigen to the subject. In some embodiments, the subject, prior to the first administration of the antibody, fragment, or molecule, has a detectable level of antibody that binds to the foreign antigen. In some embodiments, the subject, prior to the first administration of the antibody, fragment, or molecule, does not have a detectable level of antibody that binds to the foreign antigen.

In one aspect, this document features a method of inducing tolerance or reducing an immune response to a foreign antigen in a human subject. The method includes treating the subject with a regimen, the regimen includes at least one administration of an anti-CD4 antibody or a CD4-binding fragment thereof or CD4-binding molecule; and at least one administration of the foreign antigen, wherein the minimum concentration of the antibody or fragment in the blood of the subject is not less than 5 μg/mL during a tolerizing window. In some embodiments, the subject, prior to the first administration of the antibody, fragment, or molecule, has a detectable level of antibody that binds to the foreign antigen. In some embodiments, the subject, prior to the first administration of the antibody, fragment, or molecule, does not have a detectable level of antibody that binds to the foreign antigen.

In any of the methods described herein, the minimum concentration of the anti-CD4 antibody or CD4-binding fragment thereof in the blood of the subject can be from 5 μg/mL to less than 20 μg/mL during the tolerizing window.

In any of the methods described herein, the anti-CD antibody or CD4-fragment thereof, or CD4-binding molecule can be administered every other day during the tolerizing window.

In any of the methods described herein, the tolerizing window can be at least three days. For example, the tolerizing window can be at least seven days. The tolerizing window can be at least ten days. The tolerizing window can be at least fourteen days. In some embodiments, all of the days of the tolerizing window are consecutive. In some embodiments, not all of the days of the tolerizing window are consecutive.

In any of the methods described herein, the anti-CD4 antibody or CD4-binding fragment thereof, or CD4-binding molecule can be administered continuously, and wherein no more than 10 mg/kg of the antibody or the bioequivalent amount of the fragment is administered in the first 24 hour period of the regimen.

In any of the methods described herein, the first administration of the anti-CD4 antibody or CD4-binding fragment thereof can include between 0.5 mg/kg and less than 5.0 mg/kg of the antibody or the biological equivalent of the fragment. For example, the first administration of the antibody or fragment can include at least 1.5 mg/kg but less than 5.0 mg/kg of the antibody or the biological equivalent of the fragment. The first administration of the antibody or fragment can include between 1.0 mg/kg and 3.0 mg/kg of the antibody or the biological equivalent of the fragment. The first administration of the antibody or fragment can include between 50 mg and 350 mg (e.g., between 150 mg and 350 mg) of the antibody or the biological equivalent of the fragment. In some embodiments, the first administration of the antibody or fragment is the only administration of the antibody or fragment.

In any of the methods described herein, administration of the anti-CD4 antibody or CD4-binding fragment thereof, or CD4-binding molecule can include first and second administrations of the antibody or fragment or molecule, wherein the second administration is between five and eight days after the first administration.

In any of the methods described herein, after the tolerizing window, the subject has a reduced adverse immune response to the foreign antigen, wherein the subject is not otherwise immunocompromised.

In any of the methods described herein, each administration of the anti-CD4 antibody or CD4-binding fragment thereof includes at least 0.05 mg/kg but less than 5 mg/kg of the antibody or the biological equivalent of the fragment.

In any of the methods described herein, the method further can include at least one follow-up regimen, the follow-up regimen including at least one administration of the anti-CD4 antibody or CD4-binding fragment thereof or CD4-binding molecule to the subject.

In any of the methods described herein, the method further can include administering at least one compound selected from the group consisting of an antihistamine, an antiemetic, an immunosuppressant, or an anti-inflammatory.

In any of the methods described herein, the anti-CD4 antibody or CD4-binding fragment thereof can be modified to reduce binding to one or more Fc (gamma) receptors compared to the corresponding antibody or fragment without the modification.

In any of the methods described herein, the anti-CD4 antibody can be a monoclonal antibody. The antibody can be a humanized antibody. The antibody or fragment can be non-depleting. The antibody or fragment can have the six complementarity determining regions (CDRs) of TRX1. The antibody can be aglycosylated. The antibody can be designated TRX1 and contains a leucine residue at position 117. The antibody can be designated TRX1 and contains a proline residue at position 117. The antibody or fragment can have a further modification that increases its serum half-life as compared to the corresponding antibody or fragment without the further modification. The serum clearance of the further modified antibody can be reduced by at least 38% as compared to the corresponding antibody or fragment without the further modification. The further modification can increase binding of the antibody or fragment to FcRn compared to the binding of the corresponding antibody or fragment without the further modification. The antibody can be mMTRX1011A.

In any of the methods described herein, each administration of an anti-CD4 antibody or CD4-binding fragment thereof or CD4-binding molecule can be a subcutaneous administration. Each administration of an antibody or fragment or molecule can be an intravenous administration.

In any of the methods described herein, the foreign antigen can be a protein, nucleic acid, or lipid. For example, the protein can be an antibody, an enzyme, a clotting factor (e.g., factor VIII), a cytokine, a hormone, a growth factor, or a receptor. An antibody can be an anti-CD3 antibody, an anti-tumor necrosis factor (TNF) antibody, an anti-TNF receptor antibody, an anti-CD20 antibody, an anti-glycoprotein IIa/IIb receptor antibody, an anti-IL2-receptor antibody, an anti-epidermal growth factor-receptor antibody, an anti-CD52 antibody, an anti-CD11a antibody, or an anti-HER2 antibody. An enzyme can be factor IX, iduronate-2-sulfatase, alpha-L-iduronidase, alpha-glucosidase, alpha-galactosidase, arylsulfatase B, human deoxyribonuclease, or tissue plasminogen activator. A cytokine can be interferon (IFN)-alpha 2a, IFN-alpha 2b, IFN-beta 1a, IFN-beta 1b, or interleukin-2 (IL-2). A hormone can be animal insulin, recombinant human insulin, recombinant human growth hormone, gonadotropin-releasing hormone, human chorionic gonadotropin, salmon calcitonin, or recombinant human erythropoietin. A growth factor can be granulocyte-macrophage colony stimulating factor (GM-CSF), interleukin 3 (IL-3)-GM-CSF fusion protein, ciliary neurotrophic factor (NTF), or human granulocyte colony stimulating factor (G-CSF). A fusion protein can include a receptor such as TNF receptor. A nucleic acid can include a gene therapy delivery vehicle.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B are depictions of nucleotide and amino acid sequences relating to a TRX1 antibody used in the Examples below. FIG. 1A is a depiction of the nucleotide (SEQ ID NO:1) and amino acid sequence (SEQ ID NO:2) of a TRX1 antibody light chain in which the leader, FR1-FR4, CDR 1-CDR3, and the constant region are annotated. FIG. 1B is a depiction of the nucleotide (SEQ ID NO:3) and amino acid sequence (SEQ ID NO:4) of an aglycosyl TRX1 antibody heavy chain in which the leader, FR1-FR4, CDR 1-CDR3, and the constant region are annotated.

FIG. 2 is a line graph of the TRX1 serum concentration at various points during the treatment phase and washout described in Example 2. Cohort mean antibody concentration (μg/mL) was determined by enzyme linked immunosorbent assay (ELISA). Subjects received 4 doses of TRX1 antibody once daily every fourth day by intravenous infusion over two hours. Open circles, Cohort 1 (0.5 mg/kg, n=3); open squares, Cohort 2 (1.5 mg/kg, n=5); open triangles, Cohort 3 (3.0 mg/kg, n=6). The solid line represents the limit of quantitation of the ELISA assay.

FIG. 3A is a line graph of the Cohort mean absolute number of CD4+ T cells per mL of peripheral blood (cell number×10⁶/mL) at various time points. Open circles, Cohort 1 (0.5 mg/kg, n=3); open squares, Cohort 2 (1.5 mg/kg, n=5); open triangles, Cohort 3 (3.0 mg/kg, n=6). Absolute number of CD4+ T cells was calculated by multiplying the absolute number of lymphocytes determined by the complete blood count (CBC) by the percentage of CD4+ lymphocytes detected in the lymphocyte gate by flow cytometry using a CD4 domain 2 specific anti-CD4 mAb that does not compete with TRX1.

FIG. 3B is a line graph of the free CD4 molecules (TRX1 binding sites without TRX1 bound) at various time points. Free CD4 molecules were detected by whole blood staining with biotinylated TRX1. Cohort mean molecules of equivalent soluble fluorochrome (MESF) units are represented.

FIG. 3C is a line graph of the CD4 modulation by TRX1 at various time points. CD4 molecules on CD3+ T cells were detected using an anti-CD4 mAb that does not compete with TRX1 for binding. Cohort mean MESF units are represented. Data points for the three Cohorts are as described for FIG. 2A.

FIG. 4A is a line graph of the Cohort mean PhiX-specific antibody titer at the indicated days/weeks during the study. Titer is defined as the rate of phage inactivation (Kv). Subjects received three doses of PhiX during the TRX1 treatment phase with the first PhiX immunization administered immediately following the second TRX1 dose on Day 5 as indicated. Subjects were challenged twice with PhiX after the TRX1 serum level had fallen to below the level of detection. PhiX challenges were at Weeks 6 and 8 for Cohorts 1 and 2 and at weeks 7 and 9 for Cohort 3. The PhiX challenges post-last dose were different based on the dose administered to allow for complete disappearance of any free TRX1. Open circles, Cohort 1 (0.5 mg/kg, n=3); open squares, Cohort 2 (1.5 mg/kg, n=5); open triangles, Cohort 3 (3.0 mg/kg, n=6).

FIG. 4B is a series of line graphs showing the PhiX-specific antibody titer of individual subjects by Cohort (top panel, Cohort 1; middle panel, Cohort 2; bottom panel, Cohort 3).

FIG. 4C is a bar graph of the PhiX-specific IgG antibody as a percentage of the total PhiX-specific antibody at the indicated week of the study. Asterisks indicate not determined.

FIG. 5 is a graph of the Cohort mean KLH-specific antibody serum concentration (ng/mL) at the indicated week of the study. Subjects were immunized with KLH at the time of PhiX challenges, which was at weeks 6 and 8 for Cohorts 1 and 2 and weeks 7 and 9 for Cohort 3.

### DETAILED DESCRIPTION

In general, this document provides methods for inducing tolerance or reducing an immune response of a human subject to a foreign antigen. As used herein, the term “tolerance” includes partial or complete unresponsiveness of lymphocytes (CD4+ cells and/or CD8+ effector T cells and/or B cells) to receptor-mediated stimulation by antigen. A “tolerant” state or subject refers to a state or subject, respectively, in which tolerance exists. The term “tolerize” refers to inducing the partial or complete unresponsiveness and generally involves exposure of the relevant lymphocytes to, inter alia, antigen for which the relevant lymphocytes are specific. Such unresponsiveness is generally antigen-specific and persists after exposure to the tolerizing antigen has ceased to be present. For example, tolerance can be characterized by a lack of, or reduced, cytokine production (e.g., IL-2) or proliferative capacity by a tolerant T cell upon exposure to the tolerizing antigen. In one embodiment, a tolerant subject does not produce an adverse immune response, or produces a reduced adverse immune response, to the antigen over a period of time after treatment with a tolerizing agent is stopped, even when subsequently challenged with the antigen and/or when the antigen remains present in the subject, but is capable of providing an unreduced immune response against other non-crossreactive antigens.

The methods include treating the subject with a regimen that includes one or more administrations of an anti-CD4 antibody or a CD4-binding fragment thereof or a CD4-binding molecule to the subject, and one or more administrations of the foreign antigen to the subject. The methods described herein can be used for treating subjects with any disease in which the therapeutic agent that is used to treat the disease during a typical course of therapy can induce an immune response to the agent in the subject that reduces the effectiveness of the treatment. Methods described herein are particularly useful for treating subjects with a disorder characterized by the absence of a biological molecule, or the presence of a defective biological molecule such that in the subject there is reduced biological molecule activity. For example, the methods described herein can be used to treat a subject with hemophilia, hypothyroidism, growth hormone deficiency, Turner syndrome, von Willebrand disease type IIA, protein C deficiency, or a lysosomal storage disorder (LSD) such as Fabry disease, Farber disease, Gaucher disease, GM1-gangliosidosis, Tay-Sachs disease, Sandhoff disease, GM2 activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase deficiency, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia. Appropriate therapeutic agents, or plasma containing the absent agent, for some of these disorders are provided below and others would be known to those skilled in the art.

### Anti-CD4 Antibodies and CD4-Binding Fragments Thereof or CD4-Binding Molecules

The methods described herein use an anti-CD4 antibody or a CD4-binding fragment thereof or CD4-binding molecules. Such antibodies and fragments thereof are known in the art as are methods of making such antibodies and fragments. “Antibody” as the term is used herein refers to a protein that generally comprises heavy chain polypeptides and light chain polypeptides. Antigen recognition and binding occurs within the variable regions of the heavy and light chains. Single domain antibodies having one heavy chain and one light chain and heavy chain antibodies devoid of light chains are also known. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3 and IgG4) and IgM, respectively. A given antibody also comprises one of two types of light chains, called kappa or lambda, the categorization of which is based on the amino acid sequence of the light chain constant domains. IgG, IgD, and IgE antibodies generally contain two identical heavy chains and two identical light chains and two antigen combining domains, each composed of a heavy chain variable region (V_{H}) and a light chain variable region (V_{L}). Generally IgA antibodies are composed of two monomers, each monomer composed of two heavy chains and two light chains (as for IgG, IgD, and IgE antibodies); in this way the IgA molecule has four antigen binding domains, each again composed of a V_{H} and a V_{L}. Certain IgA antibodies are monomeric in that they are composed of two heavy chains and two light chains. Secreted IgM antibodies are generally composed of five monomers, each monomer composed of two heavy chains and two light chains (as for IgG and IgE antibodies); in this way the IgM molecule has ten antigen binding domains, each again composed of a V_{H} and a V_{L}. A cell surface form of IgM also exists and this has two heavy chain/two light chain structure similar to IgG, IgD, and IgE antibodies.

“Chimeric antibody” as the term is used herein refers to an antibody that has been engineered to comprise at least one human constant region. For example, one or all the variable regions of the light chain(s) and/or one or all the variable regions the heavy chain(s) of a mouse antibody (e.g., a mouse monoclonal antibody) may each be joined to a human constant region, such as, without limitation an IgG1 human constant region. Chimeric antibodies are typically less immunogenic to humans, relative to non-chimeric antibodies, and thus offer therapeutic benefits in certain situations. Those skilled in the art will be aware of chimeric antibodies, and will also be aware of suitable techniques for their generation. See, for example, Cabilly, et al., U.S. Pat. No. 4,816,567; Shoemaker, et al., U.S. Pat. No. 4,978,775; Beavers, et al., U.S. Pat. No. 4,975,369; and Boss, et al., U.S. Pat. No. 4,816,397, each of which is incorporated herein by reference in its entirety.

“Complementarity determining region” or “CDR” as the terms are used herein refer to short polypeptide sequences within the variable region of both heavy and light chain polypeptides that are primarily responsible for mediating specific antigen recognition. CDRs have been described by Kabat, et al. (1977) J. Biol. Chem. 252, 6609-6616; by Chothia, et al., (1987) J. Mol. Biol. 196:901-917; and by MacCallum, et al., J. Mol. Biol. 262:732-745, 1996, each of which is incorporated herein by reference in its entirety. There are three CDRs (termed CDR1, CDR2, and CDR3) within each V_{L} and each V_{H}.

“Fragment” or “CD4-binding fragment” as the terms are used herein in reference to an antibody refer to a polypeptide derived from an antibody polypeptide molecule (e.g., an antibody heavy or light chain polypeptide) lacking all of part of at least one chain of the corresponding antibody molecule. Antibody fragments often comprise polypeptides that comprise a cleaved portion of a full length antibody chain, although the term is not limited to such cleaved fragments. Since a fragment, as the term is used herein in reference to an antibody, encompasses fragments that comprise single polypeptide chains derived from antibody polypeptides (e.g. a heavy or light chain antibody polypeptides), it will be understood that an antibody fragment may not, on its own, bind an antigen. For example, an antibody fragment may comprise that portion of a heavy chain antibody polypeptide that would be contained in a Fab fragment; such an antibody fragment most commonly will not bind an antigen unless it associates with another antibody fragment derived from a light chain antibody polypeptide (e.g., that portion of a light chain antibody polypeptide that would be contained in a Fab fragment), such that the antigen-binding site is reconstituted. Antibody fragments can include, for example, polypeptides that would be contained in Fab fragments, F(ab′)₂ fragments, scFv (single chain Fv) fragments, diabodies, linear antibodies, multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and V_{HH} containing antibodies. It will be appreciated that “antibody fragments” or “antibody polypeptide fragments” include “antigen-binding antibody fragments” and “antigen-binding antibody polypeptide fragments.” “Antigen-binding antibody fragments” and “antigen-binding antibody polypeptide fragments” include, for example, “CD4-binding antibody fragments” and “CD4-binding antibody polypeptide fragments” and “CD4-binding fragments.”

“CD4-binding molecule” as the term is used herein refers to a peptide or protein (other than an antibody or antibody fragment) that binds to domain 1 or 2 of the CD4 receptor and mimics the signal otherwise delivered by the anti-CD4 antibody as described herein. See, Zhou and König. (2003) Cell. Signal. 15: 751-762. “CD4-binding molecule” also is referred to as “molecule” herein. A CD4-binding molecule can be, for example, a peptide that corresponds to a region of a MHC class II molecule that controls interaction with CD4, HIV envelope glycoprotein gp120 or a CD4-binding fragment thereof, interleukin-16 (IL-16) or a CD4-binding fragment thereof, or other CD4-binding molecule known in the art. See, e.g., Zhou and König. (2003), supra; and Zhou and König. (2004). Curr. Issues Mol. Biol. 6:1-16.

“Framework region” as the term is used herein refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Framework regions are sometimes referred to as “FR.” Generally, there are four FR in V_{L} and V_{H}. These are referred to, from the N-terminus to the C-terminus, as FR1, FR2, FR3, and FR4.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Those skilled in the art will be aware of humanized antibodies, and will also be aware of suitable techniques for their generation. See for example, Hwang, et al. (2005) Methods 36:35; Queen, et al. (1989) Proc. Natl. Acad. Sci. USA, 86:10029-10033; Jones, et al. (1986) Nature, 321:522-25; Riechmann, et al. (1988) Nature, 332:323-27; Verhoeyen, et al. (1988) Science, 239:1534-36; Orlandi, et al. (1989) Proc. Natl. Acad. Sci. USA, 86:3833-37; U.S. Pat. Nos. 5,225,539; 5,530,101; 5,585,089; 5,693,761; 5,693,762; and 6,180,370; and Selick, et al., WO 90/07861.

Fully human antibodies in which all segments are of human origin are also useful for the methods of this document. Methods of making fully human antibodies are known in the art. See, for example, Boerner, et al. (1991) J. Immunol., 147, 86-95, Persson, et al. (1991) Proc. Nat. Acad. Sci. USA, 88: 2432-2436; Huang and Stollar (1991) J. Immunol. Methods 141, 227-236; Hoogenboom, et al. (1998) Immunotechnology 4:1-20; Hoogenboom, et al. (2000) Immunol Today 2:371-8; Ischida, et al. (2002) Cloning Stem Cells 4(1):91-102; U.S. Pat. No. 5,798,230, and U.S. Patent Publication No. 2003-0232333.

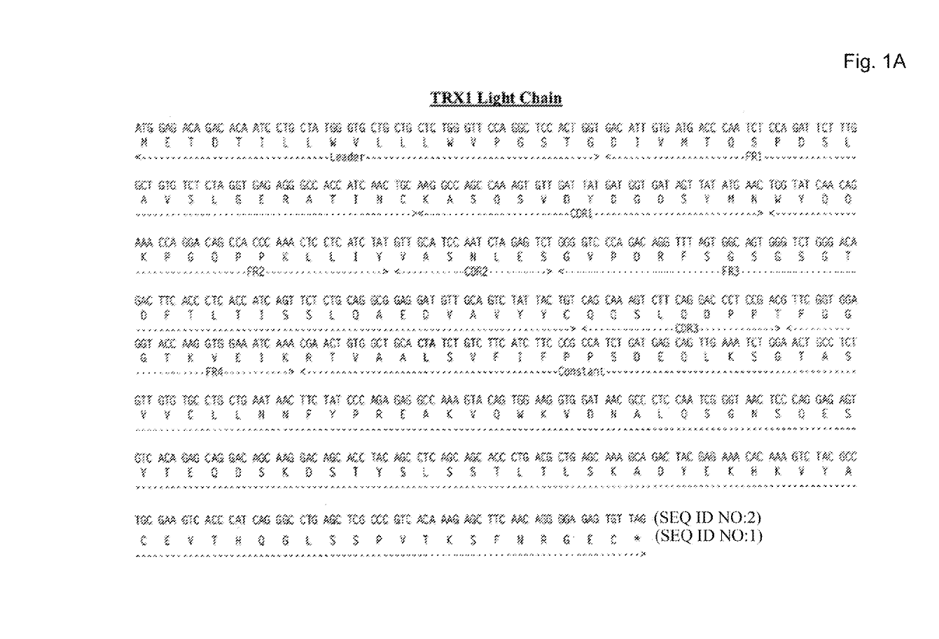

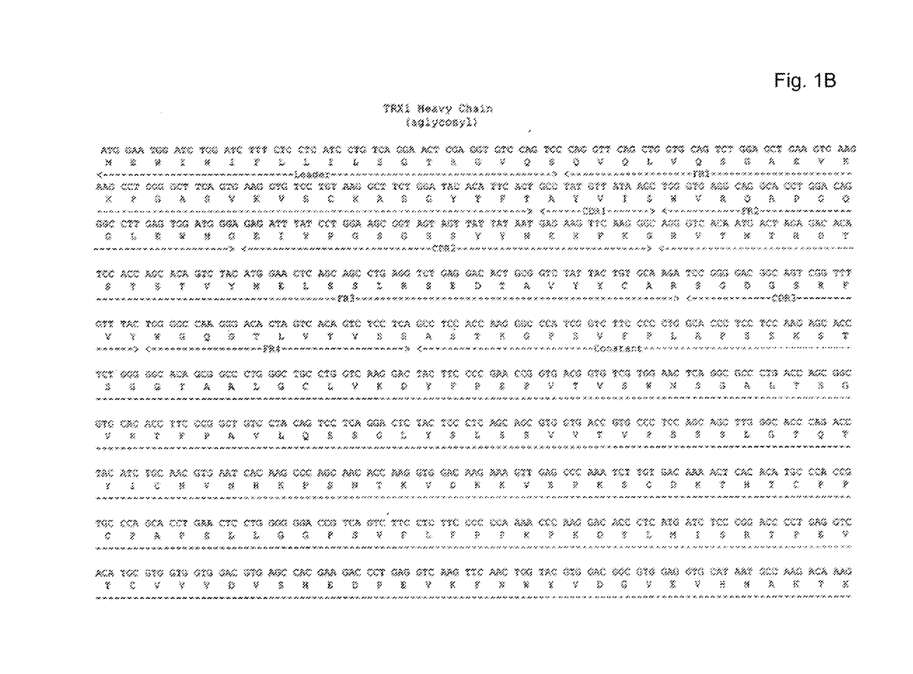

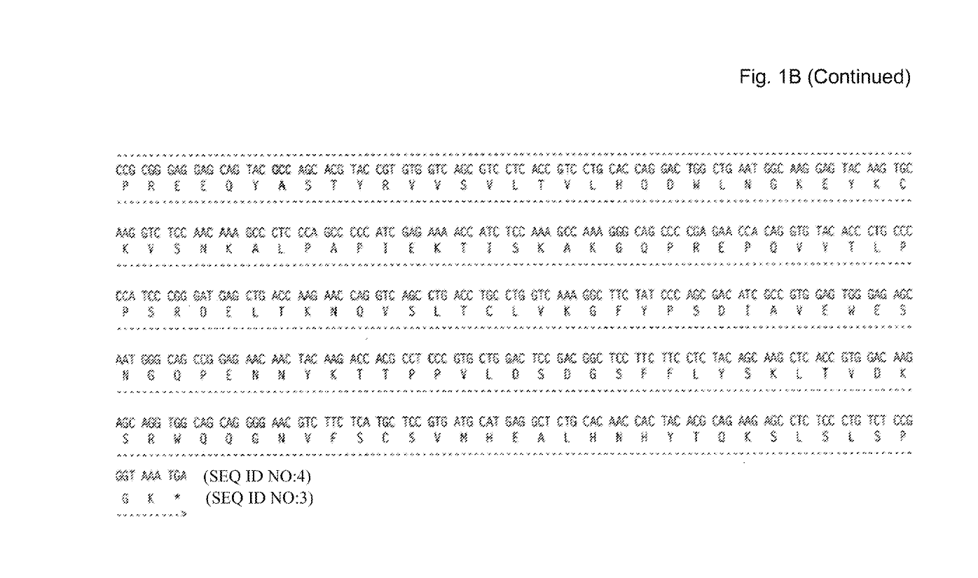

For example, an antibody to be employed in a regimen described herein can be the humanized antibody TRX1 or a CD4 binding fragment thereof, or an antibody that binds to the same domain and/or epitope or a portion thereof on human lymphocytes as humanized TRX1 antibody. A humanized anti-CD4 antibody can be designated TRX1 and include, for example, light chains and heavy chains, each containing constant regions and variable regions as depicted in FIGS. 1A and 1B, and having the amino acid sequences set forth in SEQ ID NOs: 1 and 3. See, for example, U.S. Patent Publication Nos. 20060002921 and 20040175381, the disclosures of which are incorporated by reference in their entirety.

Although a TRX1 antibody or CD4 binding fragment thereof is particularly useful, other anti-CD4 antibodies and CD4-binding fragments thereof or CD4-binding molecules can be used in the methods described herein. For example, humanized antibodies can be produced that have the same CDRs as TRX1 but a different humanized framework and/or a different human constant region. Humanized antibodies that bind to CD4 (e.g., by binding to the same domain and/or epitope as TRX1) also can be produced in which at least one amino acid in any one or more of the CDRs of TRX1 have been altered (e.g., by a conservative amino acid substitution). In such an antibody, the framework may be the same framework as TRX1 or have a different humanized framework, and/or the constant region may be the same as or different from TRX1. In some embodiments, a chimeric antibody or a murine antibody that binds to CD4 (e.g., by binding to the same domain and/or epitope as TRX1) can be produced.

The phrase “binds to the same domain and/or epitope as TRX1 humanized antibody” is intended to describe not only the TRX1 humanized antibody but also describes other antibodies, fragments or derivatives thereof that bind to the same such domain and/or epitope as the TRX1 humanized antibody. Antibodies that bind to the same domain and/or epitope as TRX1 humanized antibody can be identified using techniques known to those of ordinary skill in the art, including, for example, antibody competition assays or epitope mapping.

In some embodiments, the CD4 antibody is a CD4 antibody that has reduced effector (i.e., lytic) function as compared to human IgG₁. Examples of antibodies that would have reduced effector function include antibodies that have any one or more of the following properties: (i) an Fc portion that is aglycosylated due, for example, to a mutation in a glycosylation site (e.g., Asn-Xaa-Ser); (ii) reduced binding to the Fc receptor; or (iii) are non-lytic. For example, in one embodiment, an anti-CD4 antibody contains at least one mutation in the constant region of the heavy chain. Exemplary mutations include Leu 236 to Ala (e.g., CTG to GCG), Gly 238 to Ala (e.g., GGA to GCA), or Asn 297 to Ala (e.g., AAC to GCC). The residue numbers used herein refer to the Kabat canonical numbering system (see, e.g., Kabat, et al. (1991) Sequences of Proteins of Immunological Interest, Fifth Edition. NIH Publication No. 91-3242). Thus, for any given antibody, the residue numbers would not necessarily be the same. Those skilled in the art would be able to establish the residue numbers for a given antibody of interest from the Kabat canonical residue numbers. For example, Kabat residue 236 corresponds to position 255 of SEQ ID NO:3 (position 236 without the leader sequence in SEQ ID NO:3); Kabat residue 238 corresponds to position 257 of SEQ ID NO:3 (position 238 without the leader sequence in SEQ ID NO:3); and Kabat residue 297 corresponds to position 317 of SEQ ID NO:3 (position 298 without the leader sequence in SEQ ID NO:3).

For example, in some embodiments, the mutation at amino acid position 297 is made to produce an aglycosylated anti-CD4 antibody with reduced effector function. In some embodiments, the antibody contains two or more of such mutations. For example, an anti-CD4 antibody can be made with mutations at amino acid positions 236 and 238. Such an antibody is glycosylated, but Fc receptor and complement binding are reduced.

In some embodiments, a CD4 antibody with reduced effector function is a non-depleting CD4 antibody. As used herein, “a non-depleting CD4 antibody” is a CD4 antibody that kills or lyses more than 20% of CD4+ cells in antibody-dependent cell-mediated cytotoxicity (ADCC) or complement-mediated lysis assays. ADCC can be evaluated by labeling human peripheral blood lymphocytes (PBL) with a non-toxic intracellular dye such as a fluorescent chloromethyl derivative (e.g., CellTracker Green, C7025, Molecular Probes) then incubating the labeled PBL with the anti-CD4 antibody. After removing unbound antibody, the antibody coated PBL can be mixed with allogeneic PBL that have been activated with an anti-CD3 antibody and IL2. After incubating for a sufficient period of time (e.g., around 4 hours), propidium iodide can be used to stain dead cells and flow cytometry can be used to determine the percentage of dead cells.

Complement-mediated lysis can be evaluated by incubating human PBL or a T cell line (e.g., HUT78) with the anti-CD4 antibody followed by human heparinized plasma as a source of complement. After incubating the mixture, propidium iodide can be added to stain dead cells and flow cytometry can be used to determine the percentage of dead cells.

CD4+ cells can be quantified by various methods known in the art, including, for example, by flow cytometry. In certain embodiments, a non-depleting CD4 antibody depletes less than 25% of CD4+ cells. In certain embodiments, a non-depleting CD4 antibody depletes less than 10% of CD4+ cells. In certain embodiments, i.e., in a clinical setting, treatment with a non-depleting CD4 antibody does not result in CD4+ T cell counts below 250 cells/mm³.

In some embodiments, an anti-CD4 antibody or CD4-binding fragment thereof has a modification that increases its serum half-life as compared to a corresponding antibody or fragment thereof or molecule. For example, the anti-CD4 antibody or fragment thereof may have increased binding to FcRn and contain an amino acid modification at any one or more of amino acid residues 238, 256, 265, 272, 286, 303, 305, 307, 311, 312, 317, 340, 356, 360, 362, 376, 378, 380, 382, 413, 424 or 434 of the heavy chain (e.g., an IgG1 heavy chain), where the numbering of the residues is that of the EU index of Kabat. See, e.g., U.S. Pat. No. 6,737,056, and Shields, et al. (2001) J. Biol. Chem., 276:6591-6604. Serum half-life of an anti-CD4 antibody or CD4-binding fragment also can be increased by incorporating a serum albumin binding peptide into the antibody as disclosed in U.S. Patent Publication No. 20040001827. For example, an antibody can include a substitution of a histidine or alanine for asparagine at position 434. Kabat position 434 corresponds with position 454 in SEQ ID NO:3 (or position 435 without the leader sequence in SEQ ID NO:3). In one embodiment, the anti-CD4 antibody or CD4-binding fragment is mMTRX1011A, wherein leucine is replaced with proline at position 117, an asparagine is replaced with alanine at position 297, and an asparagine is replaced with histidine at position 434. Kabat position 117 corresponds with position 137 in SEQ ID NO:1 (position 117 in SEQ ID NO:1 without the leader sequence). Serum clearance of such an antibody is reduced by at least 38% compared to the corresponding antibody or fragment without the further modification. See, e.g., Zheng, et al. (2011) Clin Pharmacol Ther. 89(2):283-90.

In certain embodiments, one or more human framework residues can be changed or substituted to residues at the corresponding positions in the original non-human (e.g., murine) antibody so as to preserve the binding affinity of the humanized antibody to the antigen. Such a change is sometimes called “backmutation”. Certain amino acids from the human framework residues are selected for backmutation based on their possible influence on CDR conformation and/or binding to antigen. For example, residues immediately surrounding one or more CDRs can be backmutated to ensure proper spatial positioning of the CDRs. The placement of non-human (e.g., murine) CDR regions within human framework regions can result in conformational restraints, which, unless corrected by substitution of certain amino acid residues, lead to loss of binding affinity. Thus, in certain embodiments, backmutations can be made in residues that affect proper conformation of the anti-CD4 antibody or CD4-binding fragment to ensure adequate affinity to CD4.

In certain embodiments, the selection of amino acid residues for backmutation can be determined, in part, by computer modeling, using art recognized techniques. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

The selection of amino acid residues for substitution can also be determined, in part, by examination of the characteristics of the amino acids at particular locations, or empirical observation of the effects of substitution or mutagenesis of particular amino acids. For example, when an amino acid differs between a non-human (e.g. murine) framework residue and a selected human framework residue, the human framework amino acid may be substituted by the equivalent framework amino acid from the non-human binding molecule when it is reasonably expected that the amino acid: (1) noncovalently binds antigen directly, (2) is adjacent to a CDR region, (3) otherwise interacts with a CDR region (e.g., is within about 3-6 angstroms of a CDR region as determined by computer modeling), or (4) participates in the VL-VH interface.

Serum half-life of an anti-CD4 antibody or CD4-binding fragment thereof or CD4-binding molecule also can be increased by incorporating a serum albumin binding peptide into the antibody as disclosed in U.S. Patent Publication No. 20040001827.

In one embodiment, TRX1 is a humanized antibody derived from a mouse monoclonal antibody designated NSM4.7.2.4. Such an antibody contains light chain amino acid residues 132-238 of SEQ ID NO:1 (FIG. 1A) and heavy chain amino acid residues 138-467 of SEQ ID NO:3 (FIG. 1B), and light and heavy chain framework and CDR regions, in which the framework regions of the light and heavy chain variable regions correspond to the framework regions of a human light chain variable region, e.g., amino acid residues 21-43, 59-73, 81-112, and 122-131 of SEQ ID NO:1 (FIG. 1A), and framework regions of a human heavy chain variable region, e.g., amino acid residues 20-49, 55-68, 86-117, and 127-137 of SEQ ID NO:3 (FIG. 1B), and the CDRs of the light chain, e.g., amino acid residues 44-58, 74-80, and 113-121 of SEQ ID NO:1 (FIG. 1A) and the CDRs of the heavy chain, e.g., amino acid residues 50-54, 69-85, and 118-126 of SEQ ID NO:3 (FIG. 1B).

In another example, the anti-CD4 antibody or CD4-binding fragment is a modified TRX1 antibody including one or more of the following: a substitution of proline for leucine at position 117; a substitution of alanine for asparagine at position 297, and a substitution of a histidine or alanine for asparagine at position 434.

### Foreign Antigens

As used herein, the term “foreign antigen” refers to any therapeutic agent or a component of a therapeutic agent that can induce an immune response in a subject and where the immune response reduces the effectiveness of the agent to function as a therapeutic agent in the subject. The foreign antigens against which tolerance is induced in accordance with the methods described herein are not foreign antigens as present in disease-causing bacteria, fungi, viruses, etc. that infect a host, i.e., the term foreign antigen does not include a foreign antigen as part of an organism that infects a human and causes a disease or disorder.

A foreign antigen can include a protein such as an antibody or antigen-binding fragment thereof. Non-limiting examples of antibodies include an anti-CD3 antibody such as OKT3, Teplizumab, or Otelixizumab; an anti-TNF antibody such as Adalimumab (Humira®) or Infliximab (Remicade®); an anti-TNF receptor antibody such as Etanercept (Enbrel®); an anti-CD20 antibody such as Ibritumomab tiuxetan (Zevalin®) or Rituximab (Mabthera®); an anti-GPIIa/IIb-R antibody such as Abeiximab (Reopro®); an anti-IL2-R antibody such as Basiliximab (Simulect®) or Daclizumab (Zenapax®), an anti-EGF-R antibody such as Cetuximab (Erbitux®); an anti-CD52 antibody such as Alemtuzamab (Campath®); an anti-CD11a antibody such as Efalizumab (Raptiva®); or an anti-HER2 antibody such as Trastuzamab (Herceptin®).

A foreign antigen protein also can be an enzyme (e.g., an enzyme used in enzyme replacement therapy (ERT)) or a clotting factor such as factor VIII, which is used to treat Hemophilia A. For example, an enzyme can be factor IX, which is used to treat Hemophilia B; Iduronate-2-sulfatase (Elaprase®), which is used to treat Hunter syndrome (also known as Mucopolysaccharidosis II or MPS II); alpha-L-iduronidase (Adlurazyme®, laronidase), which is used to treat Mucopolysaccharidosis I H (MPS I H) (Hurler's syndrome), MPS I S (Scheie syndrome), and MPS I H-S (Hurler-Scheie syndrome); alpha-glucosidase (Myozyme®, Lumizyme®), which is used to treat Pompe's disease; alpha-galactosidase (Fabrazyme®), which is used to treat Fabry disease; or arylsulfatase B (Naglazyme®), which is used to treat Maroteaux-Lamy syndrome (MPS VI). Methods described herein can be particularly useful before or during ERT for patients with CRIM-negative (negative for crossreactive immunological material) disease such as CRIM-negative Pompe's disease (i.e., the patients lack detectable alpha glucosidase), as such patients typically develop a high titer of antibodies that neutralize the replacement enzyme and have poorer clinical outcomes.

A foreign antigen also can be part of an antiserum. Such an antiserum may be used as a replacement agent or as a new therapeutic. For example, for infectious diseases, a subject lacking, or having an inadequate level of, antibodies to a particular microbial antigen or microbial antigens can be administered an immune serum or immune pooled sera containing such antibodies to provide anti-infectious microorganism activity in the subject. For cancer, a subject lacking, or having an inadequate, anti-tumor immune response can be administered immune sera or plasma containing anti-tumor antibodies to provide the anti-tumor activity in the subject. If the missing agent occurs in serum or plasma but has not yet been purified, the subject can be administered normal donor serum or plasma containing the agent. In the case of infectious diseases and cancer, heterologous antisera (e.g., from a horse or rabbit) can be administered.

A foreign antigen protein also can be a cytokine such as interferon (IFN)-alpha 2a, IFN-alpha 2b, IFN-beta 1a, IFN-beta 1b, or interleukin-2 (IL-2); a hormone such as animal insulin, recombinant human insulin, recombinant human growth hormone, gonadotropin-releasing hormone, human chorionic gonadotropin, salmon calcitonin, or recombinant human erythropoietin; a growth factor such as granulocyte-macrophage colony stimulating factor (GM-CSF), interleukin 3 (IL-3)-GM-CSF fusion protein, ciliary neurotrophic factor (NTF) (e.g., a modified ciliary NTF such as Axokine), or human granulocyte colony stimulating factor (G-CSF); a fusion protein such as a TNF receptor fusion protein.

A foreign antigen also can be a nucleic acid or lipid. For example, a foreign antigen can be a delivery vehicle such as a vector used in gene therapy.

### Methods of Inducing Tolerance or Reducing Immune Response to a Foreign Antigen

Methods described herein include treating a subject with a regimen, where the regimen includes (i) one or more administrations of an anti-CD4 antibody or a CD4-binding fragment thereof or CD4-binding molecule to the human subject and (ii) one or more administrations of a foreign antigen to the subject. “Dosing regimen” or “regimen” as the terms are used herein, refer to the total course of treatment administered to the human subject, e.g., treatment with an anti-CD4 antibody or CD4-binding fragment thereof or CD4-binding molecule and treatment with a foreign antigen. A dosing regimen may include a given number of days of treatment, and on any day of the regimen in which dosing occurs, the dosing can be of antibody or fragment and/or antigen. For example, a regimen described herein may include administering an anti-CD4 antibody or fragment or molecule and foreign antigen to a human subject for a minimum number of days, a maximum number of days, or a specific number of days. As non-limiting examples, an anti-CD4 antibody or fragment or molecule, and/or foreign antigen may be administered to a human subject over a regimen of five days, eight days, or any number of days in between or beyond. A dosing regimen may be as short as one day, although as will be apparent from the remainder of the present specification, multiple day dosing regimens permit administration of higher amounts of antibody over a relatively short course of therapy. Regimens are generally 21 days or less (e.g., 18 days or less, 14 days or less, 13 days or less, 12 days or less, 11 days or less, 10 days or less, 8 days or less, 7 days or less, 6 days or less, 5 days or less, 4 days or less, 3 days or less, 2 days or less, or 1 day) in length. Regimens can be separated by relatively short periods of time (e.g., 5 days, 10 days, 15 days, 20 days, 25 days, 30 days, 1.5 months, 2 months, 3 months, or 4 months) or longer periods of time (e.g., 6 months, 9 months, 12 months, 18 months. 2 years, 3 years, 4 years, 5 years, 10 years, 15 years, or 20 years). Such a follow up regimen includes at least one administration of the antibody or fragment or molecule to the subject. Additionally and/or alternatively, a regimen may include a given amount of therapeutic agent administered per day. For example, an antibody or fragment or molecule and/or antigen may be administered to a human subject in a minimum amount on one or more days of the regimen, in a maximum amount on one or more days of the regimen, or in a specific amount on one or more days of the regimen.

As used herein, “tolerizing window” refers to the time period starting on the first day of a dosing regimen and extending past the end of the regimen to the first time at which no foreign antigen and/or no anti-CD4 antibody or CD4-binding fragment thereof or CD4-binding molecule is detectable (e.g., by a standard enzyme linked immunosorbent assay (ELISA) or by pharmacodynamic parameters) in the peripheral blood of the human subject undergoing the relevant dosing regimen. On any day of the regimen in which dosing occurs, the dosing can be of antibody (or fragment or molecule) and/or antigen. From this definition, it will be clear that to be in the tolerizing window, it is not required that foreign antigen and/or anti-CD4 antibody (or fragment or molecule) are detectable in the peripheral blood of the human undergoing the relevant regimen at all times from the first day to the last day of the regimen. The tolerizing window can be at least three days, five days, seven days, ten days, or at least fourteen days. After the tolerizing window, the subject has no adverse immune response or a reduced adverse immune response to the foreign antigen and is not otherwise immunocompromised.

In some embodiments, all of the days of the tolerizing window are consecutive. In some embodiments, not all of the days of the tolerizing window are consecutive. For example, a given dosing regimen may include one or more days in which the anti-CD4 antibody (or CD4-binding fragment thereof or CD4-binding molecule) and/or foreign antigen is not administered. In certain embodiments, a dosing regimen comprises one, two, three, four, five, six, seven or more days in which an antibody (or fragment or molecule) and/or antigen is not administered. In certain embodiments, the antibody (or fragment or molecule) and/or antigen is administered every other day of a dosing regimen. In certain embodiments, the antibody (or fragment or molecule) and/or antigen is administered every third day, or every fourth day.

In some embodiments, the anti-CD4 antibody or CD4-binding fragment thereof or CD4-binding molecule is administered continuously. As used herein, the term “continuous” in the context of the time in which the mean level of antibody or fragment in the blood is within a specific range of levels, means that the time the mean level is in that specific range is not interrupted by any time in which that mean level is not within that specific range of levels. For example, an anti-CD4 antibody (or fragment or molecule) can be administered continuously, wherein no more than 10 mg/kg of the antibody is administered in the first 24 hour period of the regimen. In one embodiment, no more than 5 mg/kg of the antibody is administered in the first 24 hour period. It will be appreciated that for any dose recited herein for the anti-CD4 antibody, the biologically equivalent dose for the CD4-binding fragment can be readily determined. As used herein, the biologically equivalent dose of the fragment is an amount of the fragment or molecule that achieves the same level of saturation of the CD4 binding sites on human lymphocytes that the stated amount of the corresponding whole antibody causes. Thus, in each instance in which a particular dose is recited for the antibody, it will be appreciated that the biologically equivalent dose of the fragment is also intended.

As used herein, the term “not continuous” in the context of the time in which the mean level of anti-CD4 antibody or CD4 binding fragment thereof or molecule in the blood is within a specific range of levels, means that the time the mean level is in that specific range is interrupted by some amount of time (e.g., 15 minutes, 20 minutes, 30 minutes, 45 minutes, 1 hour, 2 hours, 3 hours, 4, hours, 5 hours, 6 hours, 8 hours, 10 hours, 12 hours, 14 hours, 16 hours 18 hours, 20 hours, 24 hours 28 hours, 32 hours, 36 hours, 40 hours, 44 hours, 48 hours, 60 hours, 72 hours, 84 hours, 90 hours, or any range of time of having upper and lower limits of any of above the specifically stated times), in which that mean level is not within that specific range of levels.

### Exemplary Dosing Regimens

In certain embodiments, a treatment with an anti-CD4 antibody (or CD4-binding fragment thereof or CD4-binding molecule), and foreign antigen may be administered over a dosing regimen of one day, two days, three days, four days, five days, six days, seven days, eight days, nine days, ten days, eleven days, twelve days, thirteen days, fourteen days, or more. In certain embodiments, an anti-CD4 antibody (or fragment or molecule) and/or antigen is administered over a dosing regimen of five days. In certain embodiments, an anti-CD4 antibody (or fragment or molecule) and/or antigen is administered over a dosing regimen of eight days. In certain embodiments, an anti-CD4 antibody (or fragment or molecule) and/or antigen is administered over a dosing regimen of fifteen days. In certain embodiments, an anti-CD4 antibody (or fragment or molecule) and/or antigen is administered as a fixed dose such as by intravenous or subcutaneous administration. In other embodiments, an antibody (or fragment or molecule) and/or antigen is administered as a continuous infusion (e.g., by a microinfusion pump or slow-release patch) rather than a fixed dose. Limiting the number of days of a dosing regimen can confer practical benefits on a patient being treated. For example, limiting a dosing regimen to five days may minimize the inconvenience to a patient when that patient needs to travel to a hospital or clinic to receive anti-CD4 antibody or fragment and/or antigen treatment. Limiting the number of days in a dosing regimen can also increase patient safety since fewer hospital visits will result in fewer medical recordkeeping requirements, and thus fewer chances of making recording or filing mistakes. Limiting the number of days in a given dosing regimen can also decrease the costs associated with treatment, since the treatment provider will need to spend less total time with the patient.

In certain embodiments, an anti-CD4 antibody or CD4-binding fragment thereof or CD4-binding molecule is administered on consecutive days during a given dosing regimen. In certain embodiments, an anti-CD4 antibody or fragment thereof or molecule is not administered on consecutive days of a dosing regimen. For example, a given dosing regimen may include one or more days in which an anti-CD4 antibody or fragment thereof or molecule is not administered. In certain embodiments, a dosing regimen comprises one, two, three, four, five, six, seven or more days in which an anti-CD4 antibody or fragment thereof or molecule is not administered. In certain embodiments, an anti-CD4 antibody or fragment thereof or molecule is administered every other day of a dosing regimen. In certain embodiments, an anti-CD4 antibody or fragment thereof or molecule is administered every third day, or every fourth day, or every five to eight days during the course of the dosing regimen.

In some embodiments, the first administration of the anti-CD4 antibody is at least 0.05 mg/kg but less than 5.0 mg/kg. For example, the first administration of the antibody or fragment can be between 0.5 mg and 4.5 mg/kg, between 0.5 mg/kg and 4.0 mg/kg, between 0.5 mg/kg and 3.5 mg/kg, between 0.5 mg/kg and 3.0 mg/kg, between 0.75 mg and 4.5 mg/kg, between 0.75 mg/kg and 4.0 mg/kg, between 0.75 mg/kg and 3.5 mg/kg, between 0.75 mg/kg and 3.0 mg/kg, between 1.0 mg/kg and 4.5 mg/kg, between 1.0 mg/kg and 4.0 mg/kg, between 1.0 mg/kg and 3.0 mg/kg, between 1.5 mg/kg to 4.5 mg/kg, or between 1.5 mg/kg to 3.0 mg/kg. In some embodiments, the first administration of the antibody is at least 1.5 mg/kg but less than 5.0 mg/kg. In some embodiments, the first administration of the antibody is between 50 mg and 350 mg, e.g., 75 mg to 350 mg, 75 mg to 300 mg, 75 mg to 275 mg, 75 mg to 250 mg, 75 mg to 225 mg, 75 mg to 200 mg, 75 mg to 175 mg, 75 mg to 150 mg, 100 mg to 350 mg, 100 mg to 300 mg, 100 mg to 275 mg, 100 mg to 250 mg, 100 mg to 225 mg, 100 mg to 200 mg, 150 mg to 350 mg, 150 mg to 300 mg, 150 mg to 275 mg, or 150 mg to 250 mg. In certain embodiments, the first administration of the antibody or fragment is the only administration of the antibody or fragment.

Before the first administration of the anti-CD4 antibody or CD4-binding fragment thereof or molecule, the subject may or may not have a detectable level of antibody that binds to the foreign antigen. In subjects that do not have a detectable level of antibody that binds to the foreign antigen (i.e., are non-immunized), a lower dose of the anti-CD4 antibody (or fragment thereof or molecule) and/or foreign antigen can be used. For example, in a non-immunized subject, the first administration of antibody or fragment can range from 0.05 mg/kg to 2.0 mg/kg. In subjects that have a detectable level of antibody that binds to the foreign antigen (i.e., are not antigen naïve and may be sensitized from previous exposure), a higher dose of the antibody or fragment may be beneficial.

In certain embodiments, the regimen includes first and second administrations of the anti-CD4 antibody or CD4-binding fragment thereof or CD4-binding molecule. For example, in one embodiment, the second administration of the antibody or fragment is between 24 hours and eight days after the first administration, preferably between 2 and 8 days after the first administration and, most preferably between 5 and 8 days after the first administration.

In certain embodiments, there is at least one administration of the anti-CD4 antibody or CD4-binding fragment thereof or CD4-binding molecule at least 1 day before administration of the foreign antigen. For example, at least one dose of the antibody or fragment can be administered 2, 3, 4, 5, 6, or 7 days before administration of the foreign antigen.

In certain embodiments, each administration of the anti-CD4 antibody is at least 0.05 mg/kg but less than 5.0 mg/kg. For example, the first administration of the antibody or fragment can be between 0.5 mg and 4.5 mg/kg, between 0.5 mg/kg and 4.0 mg/kg, between 0.5 mg/kg and 3.5 mg/kg, between 0.5 mg/kg and 3.0 mg/kg, between 0.75 mg and 4.5 mg/kg, between 0.75 mg/kg and 4.0 mg/kg, between 0.75 mg/kg and 3.5 mg/kg, between 0.75 mg/kg and 3.0 mg/kg, between 1.0 mg/kg and 4.5 mg/kg, between 1.0 mg/kg and 4.0 mg/kg, between 1.0 mg/kg and 3.0 mg/kg, between 1.5 mg/kg to 4.5 mg/kg, or between 1.5 mg/kg to 3.0 mg/kg. In some embodiments, the first administration of the antibody is at least 1.5 mg/kg but less than 5.0 mg/kg.

In certain embodiments, each administration of the anti-CD4 antibody or CD4-binding fragment thereof or CD4-binding molecule is at the same amount. In other embodiments, a lower dose of the anti-CD4 antibody or fragment thereof or molecule is administered on at least one day of a dosing regimen.

In certain embodiments, during a tolerizing window that is 10 days or less, the total dose of the anti-CD4 antibody or CD4-binding fragment thereof or CD4-binding molecule administered to the subject is 30 mg/kg or less (e.g., 28 mg/kg or less, 26 mg/kg or less, 24 mg/kg or less, 22 mg/kg or less, 20 mg/kg or less, 18 mg/kg or less, 16 mg/kg or less, 14 mg/kg or less, 12 mg/kg or less, or 10 mg/kg or less). In certain embodiments, the minimum concentration of antibody or fragment thereof in the blood of the subject is not less than 5 μg/mL during a tolerizing window. For example, during a tolerizing window, the minimum concentration of the antibody or fragment thereof in the blood of the subject can range from 5 μg/mL to less than 20 μg/mL such as 5 μg/mL to 19 μg/mL, 5 μg/mL to 18 μg/mL, 5 μg/mL to 17 μg/mL, 5 μg/mL to 16 μg/mL, 5 μg/mL to 15 μg/mL, 5 μg/mL to 12 μg/mL, or 5 μg/mL to 10 μg/mL.

Any method of administration may be used to administer anti-CD4 antibodies or CD4-binding fragments thereof or CD4-binding molecules, or foreign antigen to a subject. For example, an anti-CD4 antibody or fragment thereof or molecule, and/or antigen can be administered to a patient intravenously. In some embodiments, each administration of an anti-CD4 antibody or fragment thereof or molecule is an intravenous administration. In some embodiments, an anti-CD4 antibody or fragment thereof or molecule and/or antigen can be administered to a patient by a route other than an intravenous route. For example, the anti-CD4 antibody or fragment thereof or molecule and/or antigen can be administered to a patient orally, rectally, intramuscularly, intranasally, subcutaneously, intraocularly, transdermally, by direct injection into an affected organ or tissue site, or inhaled. In some embodiments, each administration of an anti-CD4 antibody or fragment thereof or molecule is a subcutaneous administration. In some embodiments, the antibody or fragment thereof or molecule and/or antigen are administered as a continuous infusion (e.g., by a microinfusion pump or slow-release patch). In some embodiments, the patient self-administers the antibody or fragment thereof or molecule and/or antigen. Those of ordinary skill in the art will be aware of suitable routes of administration and will be able to adapt such routes of administration to any of the dosing regimens disclosed herein.

In certain embodiments, an anti-CD4 antibody or CD4-binding fragment thereof or CD4-binding molecule and/or foreign antigen is administered in a single daily dose on at least one day of a dosing regimen. In certain embodiments, an anti-CD4 antibody or fragment thereof or molecule and/or antigen is administered in a single daily dose on each day of a dosing regimen. A single daily dose of antibody or fragment thereof or molecule and/or antigen may be administered over a relatively short period of time, e.g., within a period of less than about fifteen minutes. Such embodiments minimize the hospital time and inconvenience to a patient. Alternatively, a single daily dose may be administered to a patient over a longer period of time, e.g., over a period of greater than fifteen minutes. For example, a single daily dose may be administered to a patient over a period of fifteen minutes, thirty minutes, forty-five minutes, one hour, two hours, three hours, four hours, five hours, six hours, seven hours, eight hours, nine hours, ten hours, eleven hours, twelve hours, or more. Such embodiments are useful when, for example, the patient experiences adverse side effects from administering an antibody or fragment thereof or molecule and/or antigen over a relatively short period of time. Administration of an antibody or fragment thereof or molecule and/or antigen to a patient over a period of time may be accomplished in any of a variety of ways such as, without limitation, intravenous administration.

In certain embodiments, an anti-CD4 antibody or CD4-binding fragment thereof or CD4-binding molecule is administered more than once a day on at least one day of a dosing regimen. In certain embodiments, an anti-CD4 antibody or fragment thereof or molecule is administered more than once a day on each day of a dosing regimen. For example, an antibody or fragment thereof or molecule can be administered twice, three times or four times on at least one day, or each day, of a dosing regimen. In such embodiments, there will typically be an interval between daily doses. For example, the interval between daily doses can be 1 hour, 2 hours, three hours, four hours, five hours, six hours, seven hours, eight hours, nine hours, ten hours, eleven hours, twelve hours, or more. Such embodiments are useful when, for example, the patient experiences adverse side effects from administration of the antibody or fragment thereof in a single daily dose.

In some embodiments, one or more compounds also can be administered to the human subject. For example, an antihistamine such as cyclizine, diphenhydramine (Benadryl®), dimenhydrinate (Gravol®, Dramamine®), meclozine (Bonine®, Antivert®), promethazine (Pentazine®, Phenergan®, Promacot®), hydroxyzine; an antiemetic such as a dopamine antagonist such as Alizapride®, or serotonin receptor antagonist such as Dolasetron (Anzemet®), Granisetron (Kytril®, Sancuso®), Ondansetron (Zofran®), Tropisetron (Navoban®), Palonosetron (Aloxi®), or (Remeron®); an immunosuppressant, or an anti-inflammatory compound such as an NSAID (non-steroidal anti-inflammatory drug) can be administered to the subject.

### Pharmaceutical Formulations

Anti-CD4 antibodies (or CD4-binding fragments or CD4-binding molecules) and/or foreign antigens described herein can be formulated for delivery by any available route including, but not limited to parenteral (e.g., intravenous, intradermal, or subcutaneous), oral, nasal, bronchial, ophthalmic, transdermal (topical), transmucosal, rectal, and vaginal routes. The anti-CD4 antibody (or fragment or molecule) and/or antigen containing compositions may include a delivery agent (e.g., a cationic polymer, peptide molecular transporter, surfactant, etc.) and/or a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into pharmaceutical formulations that contain an anti-CD4 antibody or fragment thereof or molecule and/or foreign antigen as described herein.

A pharmaceutical composition is formulated to be compatible with its intended route of administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection or infusion typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Pharmaceutical formulations are ideally stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the anti-CD4 antibody or CD4-binding fragment or CD4-binding molecule and/or foreign antigen in the required amount in an appropriate solvent with one or a combination of ingredients enumerated above, as required, followed by filtered sterilization. Generally, dispersions are prepared by incorporating the purified antibody or antigen binding fragment or antigen into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the anti-CD4 antibody or CD4-binding fragment thereof or CD4-binding molecule, or foreign antigen can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the anti-CD4 antibody or CD4-binding fragment thereof or CD4-binding molecule, and/or antigen, and a delivery agent are preferably delivered in the form of an aerosol spray from a pressured container or dispenser which contains a suitable propellant, e.g., a gas such as carbon dioxide, or a nebulizer. The present disclosure particularly contemplates delivery of the compositions using a nasal spray, inhaler, or other direct delivery to the upper and/or lower airway. Intranasal administration of DNA vaccines directed against influenza viruses has been shown to induce CD8+ T cell responses, indicating that at least some cells in the respiratory tract can take up DNA when delivered by this route, and the delivery agents of the invention will enhance cellular uptake. According to certain embodiments, the antibody or fragment, or antigen and a delivery agent are formulated as large porous particles for aerosol administration.

Systemic administration also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

In certain embodiments, compositions are prepared with carriers that will protect the anti-CD4 antibody or CD4-binding fragment thereof or CD4-binding molecule, and/or foreign antigen against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active anti-CD4 antibody or CD4-binding fragment thereof or CD4-binding molecule, and/or foreign antigen thereof calculated to produce the desired therapeutic effect in association with the required pharmaceutical carrier.

The anti-CD4 antibody or CD4-binding fragment thereof or CD4-binding molecule, and/or foreign antigen can be administered at various intervals and over different periods of time as required, e.g., one time per week for between about 1 to 10 weeks, between 2 to 8 weeks, between about 3 to 7 weeks, about 4, 5, or 6 weeks, etc. Those of ordinary skill in the art will appreciate that certain factors can influence the dosage and timing required to effectively treat a subject, including but not limited to the severity of the disease or disorder, previous treatments, the general health and/or age of the subject, and other diseases present. Generally, treatment of a subject with an anti-CD4 antibody or fragment thereof or molecule and/or foreign antigen as described herein can include a single treatment or, in many cases, can include a series of treatments as discussed above. It is furthermore understood that appropriate doses may depend upon the potency of the anti-CD4 antibody or fragment thereof or molecule or foreign antigen and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

Pharmaceutical formulations as described herein can be included in a container, pack, or dispenser together with instructions for administration.

Certain embodiments of methods and compositions provided herein are further illustrated by the following examples. The examples are provided for illustrative purposes only, and not to be construed as limiting the scope or content of the invention in any way.

### EXAMPLES

The TRX1 antibody used in the following examples was a humanized anti-CD4 IgG1 monoclonal antibody that binds to an epitope of domain 1 of the human CD4 receptor on human lymphocytes. The antibody was humanized by framework grafting as described by Winsor-Hines, et al. (2007). J Immunol. 173(7):4715-4723. A single amino acid substitution, N297A, in the heavy chain Fc region was introduced to eliminate the only site for N-linked glycosylation and thereby abrogate Fc receptor binding and complement fixation. TRX1 was produced by genetically engineered CHO cells in hollow fiber bioreactors.

Modulation of immune responses by TRX1 was evaluated using two neoantigens: the non-pathogenic bacteriophage PhiX174 (referred to interchangeably herein as “PhiX” or “PhiX174”) and BCI-ImmuneActivator™ keyhole limpet hemocyanin (KLH). These neoantigens were selected because subjects could be expected to have no pre-existing immunity to them and their safety and tolerability were well established. PhiX is often used to examine T cell-dependent humoral immune responses that may be compromised due to experimental manipulation resulting in immunodeficiency. See, Andrews, et al. (1997) Blood, 90(4):1701-1708; Bearden, et al. (2005) Am J Transplant, 5(1):50-57; and Krueger, et al. (2008) J Invest Dermatol. 128(11):2615-2624.

### Example 1

### Clinical Study Design

Seventeen human subjects with refractory cutaneous lupus erythematosus (CLE) were enrolled in a Phase 1b, multicenter, open-label, dose-escalation study to investigate the safety, tolerability and pharmacokinetics (PK) of TRX1 administered by intravenous infusion. The Investigational New Drug (IND) application was FDA (Food and Drug Administration) approved, and the study was approved by the institutional review board at each site. Written informed consent was obtained from all subjects before participation in the study.

This Phase 1b study in human subjects with refractory CLE evaluated the immunomodulatory activity and safety of TRX1 and expanded on findings obtained in the earlier single-dose, dose-escalation Phase I study (Ng, et al. (2006) Pharm Res., 23(1):95-103). Three consecutive dosing Cohorts were enrolled (0.5, 1.5 and 3.0 mg/kg/dose; Cohorts 1, 2 and 3, respectively). TRX1 infusions were administered once daily every fourth day over 2 hours on days 1, 5, 9, and 13 for a total of 4 doses. Eleven of 17 subjects including all subjects in Cohorts 1 and 3 had chronic discoid lupus erythematosus (DLE). All completed the core study assessments through week 14. Three subjects from Cohort 2 received only 1 of 4 planned doses of TRX1, therefore, additional subjects were enrolled into Cohort 2 than originally planned. Demographics and baseline characteristics are shown in Table 1.

TABLE 1
Demographic and baseline characteristics of subjects

| Characteristic | Cohort 1 | | Cohort 2 | | Cohort 3 | |
|---|---|---|---|---|---|---|
| Subject age (years) - Mean (SD) | 36.0 | (8.72) | 46.1 | (8.24) | 43.5 | (9.44) |
| Subject weight (kg) - Mean (SD) | 63.20 | (10.86) | 67.99 | (9.64) | 76.92 | (22.80) |
| Gender - N (%) | | | | | | |
| Male | 0 | (0) | 2 | (25.0) | 0 | (0) |
| Female | 3 | (100.0) | 6 | (75.0) | 6 | (100.0) |
| Ethnicity - N (%) | | | | | | |
| Hispanic/Latino | 0 | (0.0) | 1 | (12.5) | 0 | (0.0) |
| Not Hispanic/Latino | 3 | (100.0) | 7 | (87.5) | 6 | (100.0) |
| Race - N (%) | | | | | | |
| Asian | 0 | (0.0) | 1 | (12.5) | 0 | (0.0) |
| Black/African American | 1 | (33.3) | 1 | (12.5) | 2 | (33.3) |
| Native Hawaiian/Other Pacific | 1 | (33.3) | 0 | (0.0) | 0 | (0.0) |
| Islander | | | | | | |
| White | 1 | (33.3) | 5 | (62.5) | 4 | (66.7) |
| Other | 0 | (0.0) | 1 | (12.5) | 0 | (0.0) |
| Time since lupus diagnosis (years) - | 7.0 | (4.58) | 9.4 | (10.35) | 6.0 | (2.53) |
| Mean (SD) | | | | | | |
| Lupus erythematosus (LE) | | | | | | |
| diagnosis - N (%) | | | | | | |
| SLE with cutaneous | 0 | (0.0) | 1 | (12.5) | 0 | (0.0) |
| involvement | | | | | | |
| Subacute cutaneous LE | 0 | (0.0) | 2 | (25.0) | 0 | (0.0) |
| Chronic cutaneous LE | 0 | (0.0) | 3 | (37.5) | 0 | (0.0) |
| Discoid LE | 3 | (100.0) | 2 | (25.0) | 6 | (100.0) |

TRX1 was generally well tolerated. Fourteen of 17 subjects (82.4%) received all 4 doses of TRX1. Three subjects in Cohort 2 had adverse events (AE) that led to discontinuation after the first dose of study drug: one because of a rash, one because of an injection site extravasation, and one because of a serious AE considered unrelated to study drug (acute coronary syndrome in a subject with significant underlying coronary artery disease). There were no deaths. Fifteen (88.2%) subjects reported at least one AE after dosing with TRX1. The most commonly reported AEs were pruritus (23.5%), rash (23.5%), decreased lymphocyte count (17.6%), decreased neutrophil count (17.6%), and headache (17.6%). Adverse events were not dose related.

Anti-TRX1 antibodies were not detected in the subjects prior to the first administration. Sera from all subjects were assessed for anti-TRX1 antibodies using a bridging ELISA capable of identifying antibodies to the entire TRX1 molecule. Plates were coated with TRX1 at a concentration of 5 ng/ml (50 μl/well) and incubated overnight at 4° C. Plates were washed 3 times with PBS containing 0.05% Tween 20 and blocked with 200 μl of PBS with 1% BSA for 2 hours at 37° C. Serum samples and a standard (goat anti-human IgG) were transferred to a TRX1 coated plate that was incubated for 1.5 to 2 hours at 37° C. Plates were washed 3 times before biotin-conjugated TRX1 was added to each well (50 μl/well) and incubated for 1 hour at ambient temperature. Plates were washed again. Horseradish peroxidase (HRP) labeled streptavidin (50 μl/well) was added and plates were incubated for another hour at ambient temperature before they were washed again. Development substrate was added to all wells (50 μl/well) and incubated at ambient temperature for 5-10 minutes. After the reaction was stopped, the optical density (OD) was assessed with a Supermax Plus plate reader (Molecular Devices Corp., wavelength of 490 nm) running SOFTmax PRO data acquisition and analysis software. Results for test samples were reported by extrapolation from the standard curve. The limit of quantification for the ELISA was 3.13 μg/ml.

Pruritic rash was reported in five subjects, three in Cohort 2 and two in Cohort 3. The time of onset varied from 6 hours to more than 30 days after the first infusion of antibody. All rashes were assessed as mild to moderate and resolved. Pruritic rash was observed in a previous clinical study with TRX1 for treatment of rheumatoid arthritis and has been reported with other depleting and non-depleting anti-CD4 antibodies. See, Choy, et al. (2002) Rheumatology (Oxford). 41(10):1142-1148; Choy, et al., (2000) Rheumatology (Oxford). 39(10):1139-1146; and Mason, et al. (2002) J Rheumatol. 29(2):220-229. One subject in Cohort 3 experienced a mild rash on the lower left leg 31 days after the first infusion that was assessed as unlikely to be related to TRX1. The subject tested seropositive for varicella zoster virus (VSV) with elevated IgM and IgG titers consistent with a herpes zoster reactivation.

The levels of cytokines (interleukin (IL)-2, IL-5, IL-6, IL-13, tumor necrosis factor (TNF)-α, transforming growth factor (TGF)-β1, interferon (IFN)-γ, and tryptase) varied greatly between subjects. A validated ELISA for each cytokine was carried out by Esoterix Laboratories, Calabasas, Calif. A transient increase in IL-6 and TNF-α was observed in some subjects after the first and/or last infusion of TRX1 (data not shown). However, increases in cytokine levels were not associated with signs or symptoms typical of cytokine release and were not dose-dependent. Furthermore, there were no instances of cytokine-release syndrome.

A transient improvement from baseline in CLASI scores was observed in all cohorts. The CLASI is a validated measurement instrument for LE used in clinical trials, and has separate scores for damage and activity. See, e.g., Klein, et al. (2011) Arch Dermatol., 147(2):203-208 (2011). The CLASI, used here, is used for subjects with only dermatological manifestations. Cohorts 1 and 2 showed an improvement of 22% and 32% from baseline, respectively, at 8 weeks. An improvement from baseline of 27% and 22% was also observed at 14 weeks in Cohorts 2 and 3, respectively. A change from baseline of greater than 20% is considered clinically significant. No durable improvement from baseline in CLASI scores was observed in any of the Cohorts.

Serum samples were collected for PK analysis at the pre-dose visit (day 0), and on day 1 at 1, 2, 4, 8, and 24 hours after the start of infusion. On days 5 and 9 (second and third infusions), serum samples were collected prior to the start of infusion and immediately after the 2 hour infusion was completed. On day 13 (fourth infusion), TRX1 elimination kinetics was assessed using serum samples taken before the start of infusion and at 1, 2 (end of infusion), 4, 8, and 24 hours after the start of infusion. Single serum samples were collected on days 14, 19, 22, 26, 33, and 40. Whole blood samples were collected for pharmacodynamic (PD) analyses at screening, baseline (day 0), immediately prior to infusion, 2, 3 and 8 hours after the start of infusion, on days 14, 22, and at weeks 4, 6, 8, 10, 12, and 14.

All standard statistical analyses were performed using the SAS System®, Version 8.02. Unadjusted 1-way ANOVAs were performed by Cohort on mean phage inactivation activity level and titer following exposure to PhiX and KLH, respectively. The least-squared means test was used to evaluate pair-wise differences between Cohorts.

### Example 2

### Pharmacokinetic (PK) Analysis

TRX1 serum concentration was determined by ELISA. Plates were coated with 50 μl/well of soluble human CD4 (Affinity Bioreagents) at a concentration of 2 μg/ml in PBS and incubated overnight at 2-8° C. Plates were washed with PBS containing 0.05% Tween 20 and blocked for approximately 2 hours with 200 μl/well of PBS plus 1% BSA. Standards and samples (50 μl/well) were transferred to the soluble CD4 coated plates and were incubated for 1 hour at room temperature. After washing, secondary antibody (peroxidase-conjugated goat anti-human IgG F(ab)′2, Jackson ImmunoResearch) was added, and plates were incubated for 1 hour at room temperature. Plates were washed and developed for approximately 5 minutes at room temperature. After the reaction was stopped, OD was assessed with a SpectraMax Plus plate reader (MDS Analytical Technologies, Sunnyvale, Calif.) at a wavelength of 490 nm. OD values were imported into SOFTmax® PRO (version 4.3.1 LS, MDS Analytical Technologies), and concentrations were determined from a standard curve. The limit of quantification was 4 μg/ml.

PK parameters of TRX1 were estimated using non-compartmental techniques (WinNonlin® Pro software, version 4.0.1 [Pharsight Corporation, Mountain View, Calif.]). Cmax and time to Cmax (Tₘₐₓ) were the observed values. Exposure from time zero to last observable time (AUCₗₐₛₜ) was estimated using the linear-log rule. t_{1/2} was calculated using the terminal linear portion of the log concentration-time curve.

The geometric mean was provided for dose-dependent parameters. Graphical assessments were used to explore PK linearity. When sufficient data were available, dose proportionality was assessed using ANOVA and linear regression techniques. The following model was used to assess the dose-response relationship:

logₑ(Parameter)=a+b*logₑ(dose),

where ‘a’ is the intercept and ‘b’ is the slope. This is referred to as a power model because after exponentiation:

Parameter=α*dose^{b}

where the estimate of b is a measure of dose proportionality and α=e^{a}. In this model, the actual dose administered was used. Dose proportionality was confirmed if the 95% confidence interval (CI) constructed for the estimate of ‘b’ included a value of 1.0. The fold-increase in exposure expected for a doubled dose was estimated as 2^{b} with 95% CI (2^{bL}, 2^{bU}) where bL and bU represent the 95% confidence limits for b.

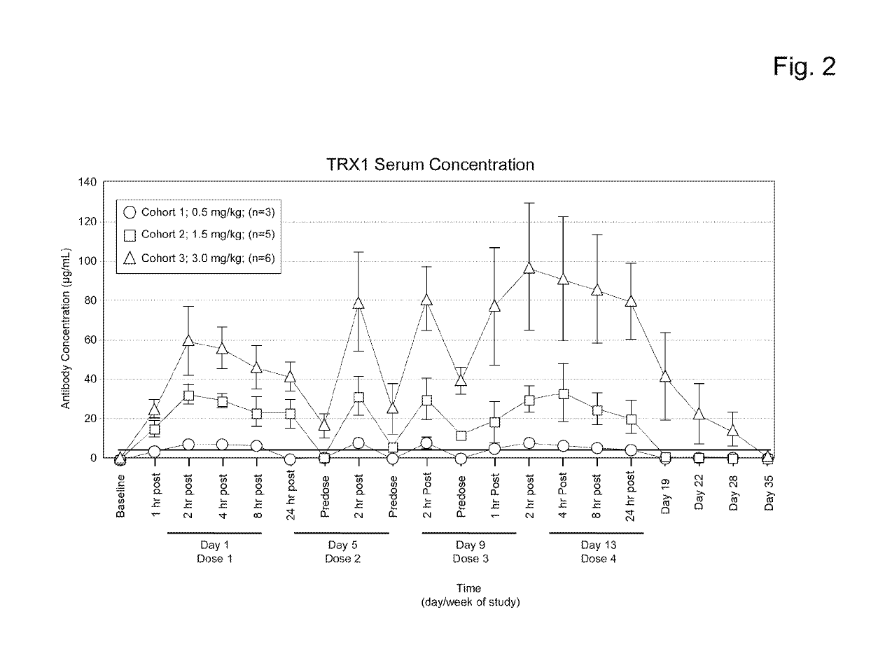

TRX1 was detectable in serum by the end of the first 2-hour infusion for Cohort 1 and at 1 hour after the start of the first infusion in Cohorts 2 and 3 (FIG. 2). Twenty four hours after the first dose the serum concentration of TRX1 was 22.5±7.1 μg/ml and 41.6±7.5 μg/ml in Cohorts 2 and 3, respectively, but had fallen below the level of quantitation (BLQ) in Cohort 1. In fact, TRX1 serum level was BLQ prior to the start of each of the three subsequent doses in Cohort 1. Although TRX1 serum level fell to BLQ in Cohort 2 prior to the second dose, both Cohorts 2 and 3 showed accumulation of TRX1 with mean serum concentration minima gradually increasing after each dose. With the fourth dose (day 13), TRX1 mean serum levels in Cohort 1 were detectable up to 24 hours post-dose, while levels in Cohort 2 were detectable up to 6 days post-dose. In Cohort 3, TRX1 was detectable up to 9 days after the last dose in all 6 subjects and as much as 12 days post-dose in 3 subjects. TRX1 serum levels were no longer detectable beyond days 14, 19, and 35 for Cohorts 1, 2, and 3, respectively.

### Example 3

### Pharmacodynamic (PD) Analysis

Percentages of CD3⁺, CD4⁺, CD8⁺, and CD19⁺ cells was measured using flow cytometry according to a validated procedure (ICON Laboratories, Farmingdale, N.Y.). Blood was collected into a heparinized BD Vacutainer® and held at room temperature until analysis. Washed whole blood cell samples were mixed with antibodies and incubated in the dark at room temperature for 20 minutes, after which lysis reagent was added. Samples were incubated, washed, and resuspended in 3% paraformaldehyde. Analysis was performed within 24 hours using a BDIS FACSCalibur Flow cytometer collecting 50,000 gated events.

Molecules of equivalent soluble fluorochrome (MESF) values were determined by comparing the fluorescence intensity signal from a microbead standard to the signal from the sample. Absolute counts for each lymphocyte subset were calculated by multiplying the absolute number of lymphocytes per milliliter obtained from a CBC drawn at the time of the flow sample by the percentage of lymphocytes in the lymphocyte flow cytometry gate bearing the CD marker of interest. Cell-bound TRX1 was detected on CD4⁺ T cells and monocytes with an anti-human IgG antibody. The MESF of the anti-human IgG was used to quantify the amount of cell-bound TRX1.

CD4 modulation was determined using a non-competing, domain 2 specific anti-CD4 (M-T441 Ab, Ancell, Bayport, Minn.). MESF values for the anti-CD4 antibody was used to quantify the number of CD4 sites on CD4+ T cells and monocytes. To assess saturation, free TRX1 binding sites were detected by staining with biotinylated TRX1. The MESF value of bound biotinylated TRX1 was directly proportional to the availability of free TRX1 binding sites and indicative of saturation of CD4. The MESF was reported for each Cohort.

Cell surface expression of CD4 was assessed with a domain 2 specific mAb that is non-competitive with TRX1 binding. CD4 saturation was determined as a function of free CD4 sites on circulating lymphocytes. The number of lymphocytes in peripheral blood was determined by multiplying the absolute lymphocyte count obtained from a CBC drawn at the time of the flow sample by the percentage of CD4⁺ lymphocytes detected in the lymphocyte gate by flow cytometry using a domain 2 specific anti-CD4 mAb that is non-competitive with TRX1.

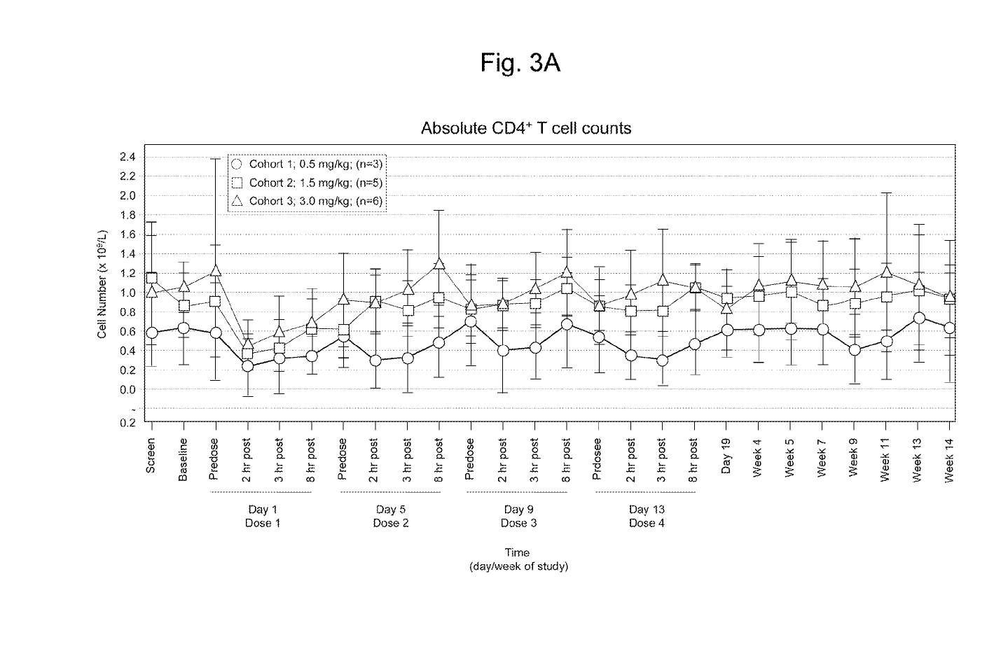

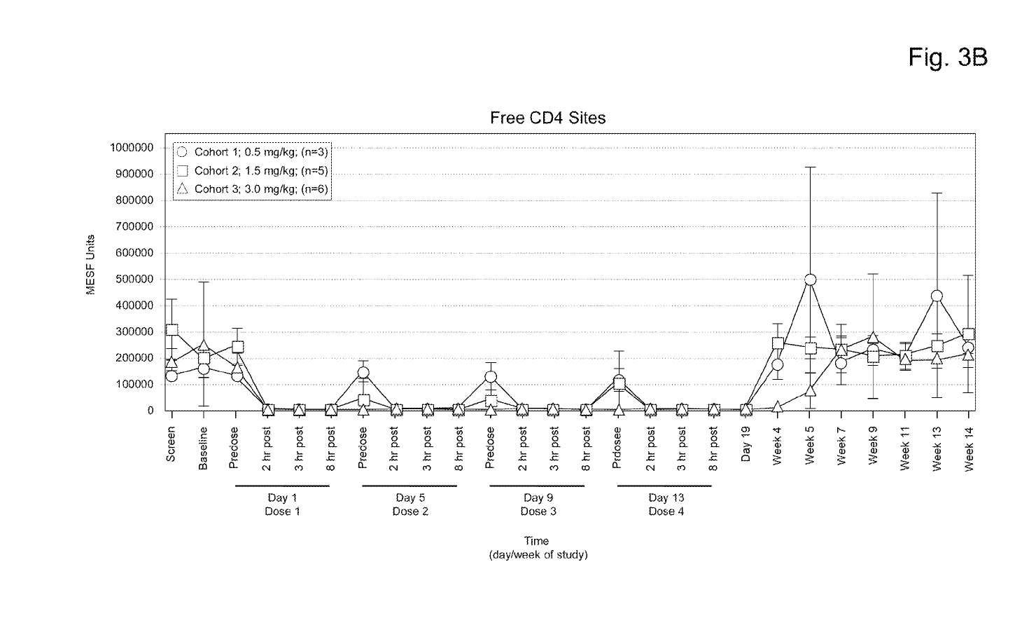

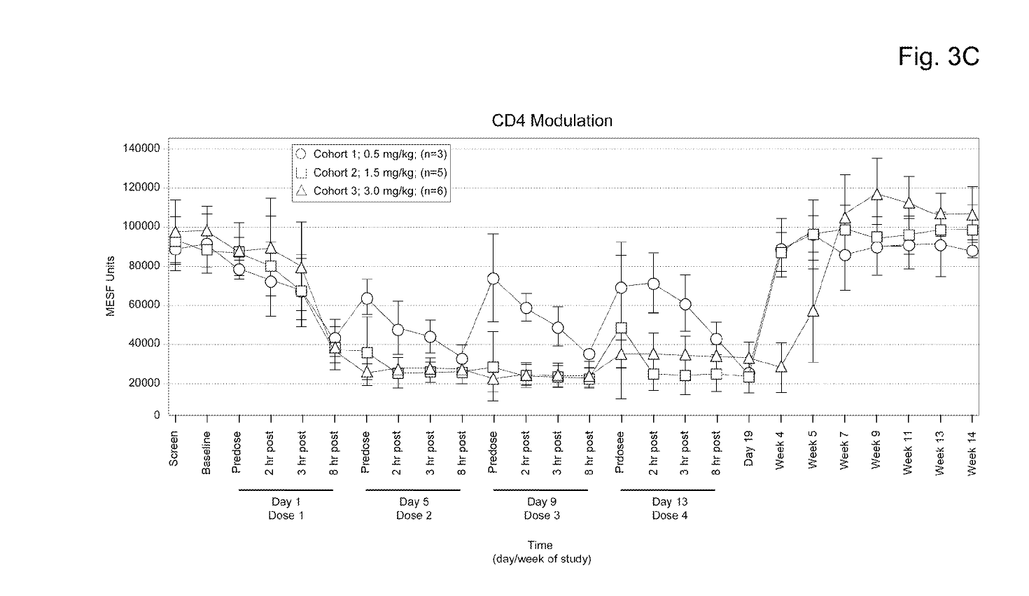

Circulating CD4⁺ T cell counts were transiently reduced after the first dose of TRX1 but returned to baseline before the second dose in all 3 Cohorts (FIG. 3A). In Cohort 1, CD4⁺ T cells decreased after the second and third doses of TRX1 but returned to baseline before subsequent doses and by 4 days after the last dose (day 19). Decreases in CD4⁺ T cells were not observed in Cohort 2 or 3 after the second through fourth doses. There was no effect of TRX1 on CD8⁺ T cell or CD14⁺ monocyte counts (not shown). In some subjects a small, but transient, reduction in the number of circulating CD19⁺ B cells was observed immediately after dosing (not shown). B cells were highly variable and the changes were not considered significant. Levels of CD4⁺CD69⁺, CD8⁺CD25⁺, and CD8⁺CD69⁺ T cells were also variable and exhibited no consistent trends (not shown).

Free CD4 sites decreased immediately after dosing with almost complete saturation at the end of the first infusion that was still evident 8 hours after the start of infusion in all Cohorts (FIG. 3B). In Cohort 1, free CD4 sites returned to baseline levels prior to doses 2 through 4 and by week 4 after the last dose. Greater saturation of CD4 was maintained in Cohort 2, but free sites were detectable (25-50% of baseline) prior to subsequent doses and returned to baseline levels by week 4. In contrast, complete saturation of CD4 sites was maintained throughout the dosing period in Cohort 3 with free sites first detectable at week 5 with return to baseline by week 7. Thus, the degree of CD4 saturation after administration of TRX1 was dose-dependent and temporally associated with serum levels.

In Cohort 1, partial down-modulation occurred after the first dose (FIG. 3C) and was maintained throughout the dosing period although with partial recovery after each dose. CD4 surface expression returned to baseline levels by week 4. Maximum CD4 down-modulation of 75% was observed after the first infusion in Cohort 3 and after the second infusion in Cohort 2. This level of modulation was sustained throughout most of the dosing period with some degree of recovery evident in both Cohorts 2 and 3 prior to the last dose. This level of maximum modulation is consistent with a number of previous studies with anti-CD4 antibodies for treatment of rheumatoid arthritis. See, Mason, et al., 2002, supra; and Choy, et al. (1996) Arthritis Rheum. 39(1):52-56. Surface expression of CD4 returned to baseline by week 4 in Cohort 2 and week 7 in Cohort 3.

### Example 4

### Response to Neoantigens PhiX174 and KLH

Subjects were immunized with PhiX174 during the course of TRX1 administration to evaluate potential immunosuppressive effects of the antibody and to determine if administration of antigen during a relatively short course of TRX1 exposure would induce durable antigen-specific tolerance or hyporesponsiveness to foreign antigen in human subjects with ongoing inflammatory autoimmune disease.

Bacteriophage PhiX174 is an investigational product covered under US FDA BB-IND 714 and is manufactured in the laboratory directed by Dr. Hans Ochs at the University of Washington in Seattle, Wash. (USA). PhiX174 has been studied as a T cell-dependent antigen and designated by the WHO Committee of Primary Immunodeficiency Diseases as a standard antigen for the assessment of the immune response in humans.

After TRX1 was no longer detectable in the serum, subjects were challenged with PhiX to assess the long-term immunomodulatory effects of PhiX exposure under the cover of CD4 blockade. PhiX was administered according to a schedule different from the dosing schedule used for assessment of immune function. PhiX (1×10¹¹ PFU/ml) was administered i.v. at a dose of 0.022 ml/kg (2×10⁹ PFU/kg) over 20 to 30 seconds. Antibody activity was determined using a standard phage neutralizing assay, and activity was expressed as the rate of phage inactivation (Kv).

All subjects were immunized with PhiX except for 3 subjects in Cohort 2 who received only 1 dose of TRX1. PhiX was administered during treatment with TRX1 on days 5, 9, and 13. This was followed with challenge doses of PhiX after TRX1 was no longer detectable in serum. Challenge doses of PhiX were administered at weeks 6 and 8 for Cohorts 1 and 2 and weeks 7 and 9 for Cohort 3.

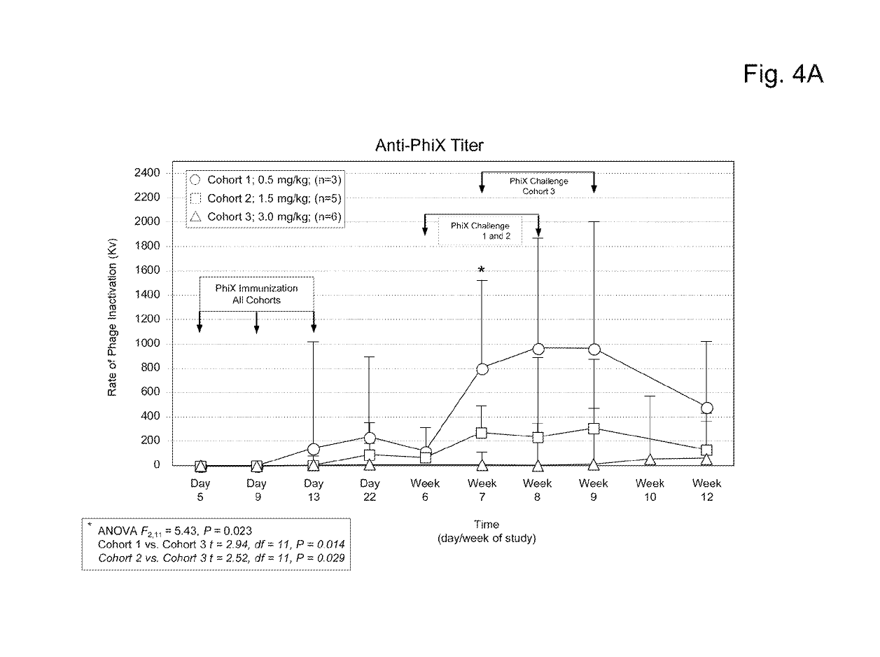

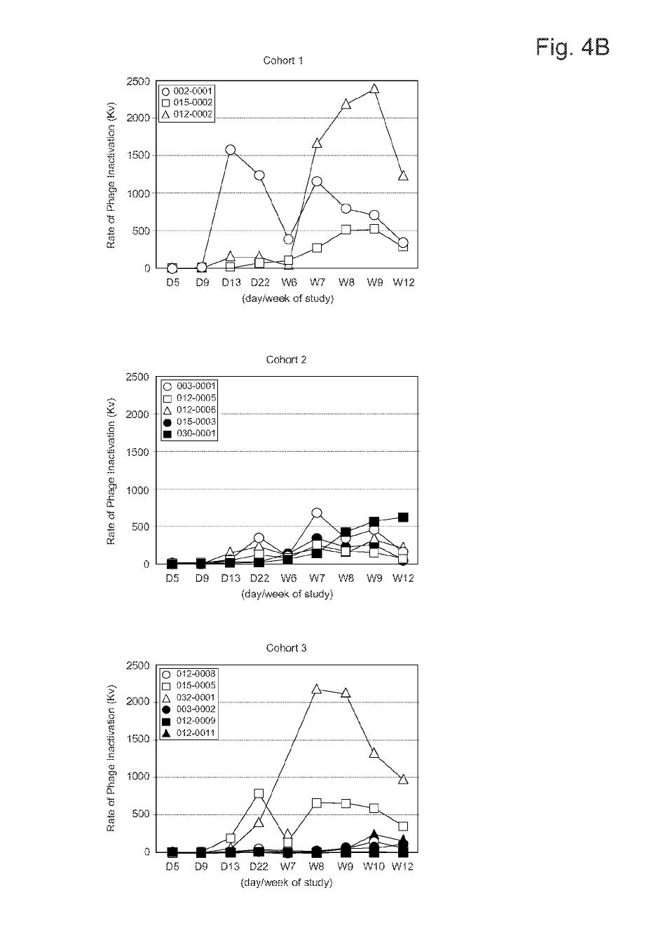

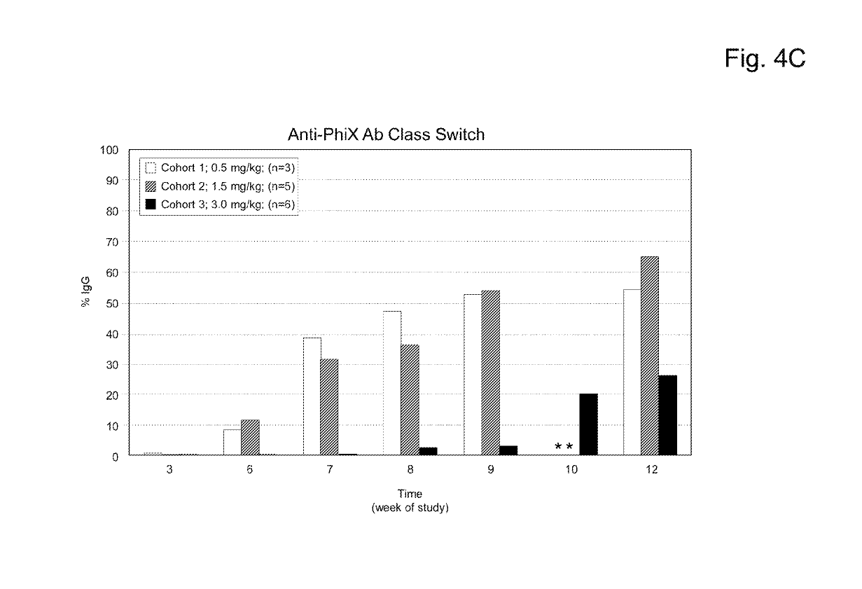

Subjects in Cohort 1 generated a primary antibody response to PhiX during the TRX1 treatment phase with peak Cohort mean Kv=241.6±654.3 (n=3) on day 22 (FIG. 4A). A more robust secondary response was generated with PhiX challenge, peak responses occurring at weeks 8 and 9 with a mean Kv of 966.6±902.5 and 966.3±1035.7, respectively. Antibody responses to PhiX were less pronounced during TRX1 treatment in Cohorts 2 and 3 with day 22 Kv of 99.2±139.5 (n=5) and 33.0±321.8 (n=6), respectively, indicating TRX1-mediated suppression of the primary immune response at the higher doses. The secondary responses generated to PhiX challenges were substantially lower in Cohorts 2 and 3 in comparison with Cohort 1 indicating a degree of hyporesponsiveness at the 2 higher doses of TRX1. The peak secondary response for Cohort 2 was observed at week 9 with a mean Kv=316.2±159.0. For Cohort 3 the peak response to PhiX challenge occurred at Week 10 with a mean Kv=78.1±499.6. Differences in mean Kv between cohorts were significant only at week 7 (ANOVA F2,11=5.43, P=0.023), prior to administration of the first PhiX challenge to Cohort 3 (Cohort 1 vs. Cohort 3 t=2.94, df=11, P=0.014; Cohort 2 vs. Cohort 3 t=2.52, df=11, P=0.029). Differences between Cohorts 1 and 2 were not statistically significant.

The dose-dependent hyporesponsiveness to PhiX challenge is particularly evident upon examination of individual subject responses in each Cohort (FIG. 4B). All subjects in Cohort 1 responded to PhiX with a maximum Kv>500 (Kv range, 523-2406). Anti-PhiX responses in Cohort 2 were substantially diminished with only two of five subjects responding with a maximum Kv above 500 (Kv range, 256-681). Similarly, in Cohort 3 two of six subjects responded with a maximum Kv>500 (Kv range, 0.3-2164). However, the two highest responding subjects, 015-0005 and 032-0001, showed elevated anti-viral titers to VSV and CMV/EBV, respectively. Excluding these two subjects, the remaining four subjects responded with a maximum anti-PhiX titer of Kv=254.

In addition to total (IgG+IgM) neutralizing anti-PhiX titers, the level of phage specific IgG antibody was assessed as a measure of T cell help, given its necessity for isotype switching (FIG. 4C). Prior to the first PhiX challenge at week 6, IgG represented 8.3% and 11.5% of the total phage specific antibody in Cohorts 1 and 2, respectively. One week after the second and final challenge (week 9) the percent of phage specific IgG increased to 52.6% and 53.4%, respectively. In Cohort 3, IgG represented only 0.4% of the total phage specific antibody prior to the first PhiX challenge at week 7. The percentage of IgG increased to 20.1% by one week after the second PhiX challenge and to 26.5% two weeks later (week 12). Almost all of the increase in phage specific IgG, as indeed, almost all of the total anti-PhiX phage specific titer was generated by responses in only 2 of the 6 subjects in Cohort 3 (not shown).

To summarize, in subjects with CLE, TRX1 suppressed the primary humoral immune response to PhiX antigen immunization in an antigen- and dose-dependent fashion. The highest dose of TRX1 resulted in hyporesponsiveness to repeated challenges with PhiX well after the antibody had been cleared. The hyporesponsiveness of the high dose Cohort compared to the lower dose Cohorts, as measured by a lower total neutralizing phage specific antibody titer, was also reflected in the isotype composition of the PhiX specific antibody. In the high dose Cohort four of six patients demonstrated a negligible antibody isotype switch to IgG, while two others mounted responses accounting for almost all of the IgG and total anti-PhiX antibody response of the Cohort. Moreover, these 2 subjects showed elevated anti-viral titers during the course of TRX1 treatment suggesting that rather than inducing inadvertant tolerance or hyporesponsiveness to a pathogen, infection during anti-CD4 mAb treatment may abrogate such induction. This response is consistent with observations using infection to prevent tolerance induction in murine models and similar observations using anti-CD4 antibody in primates.

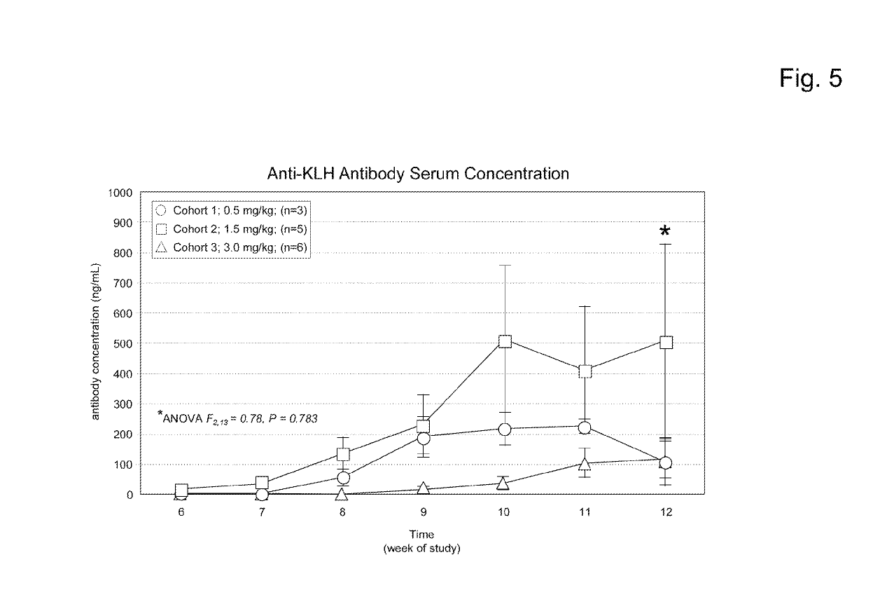

The antigen specificity of any long-term hyporesponsiveness was assessed by immunizing all subjects with a second neoantigen, keyhole limpet hemocyanin (KLH), given after TRX1 was no longer detectable in serum (FIG. 5). KLH (BCI-ImmuneActivator™) is an investigational product covered under US FDA BB-IND 4250 and manufactured by Intracel (Frederick, Md.). KLH was used to assess whether TRX1 had any suppressive effect on the subject's ability to mount a humoral immune response to a T cell-dependent neoantigen that was not present during the TRX1 dosing period. The product was supplied in a 3-ml sterile glass vial with a volume of 1.2 ml at a concentration of 5 mg/ml.

Primary immunization with KLH (week 6 for Cohorts 1 and 2; week 7 for Cohort 3) was followed by 2 KLH challenges (weeks 7 and 8 for Cohorts 1 and 2; weeks 8 and 9 for Cohort 3). All Cohorts mounted an immune response to KLH with no significant differences among groups at week 12 (ANOVA F2,13=0.78, P=0.783). Thus, there was no evidence of non-specific immune suppression to a neoantigen administered after TRX1 clearance.

The antigen specificity of the hyporesponsiveness was assessed by immunizing all subjects with the neoantigen KLH after TRX1 had been cleared from the circulation. Although the response to KLH was more variable, there was no statistically significant difference between Cohorts in the serum concentration of anti-KLH antibody, and responses to KLH when compared to historical controls. This indicates that TRX1 treatment did not result in long-term non-specific immune suppression.

In previous clinical studies with non-depleting CD4 antibodies in rheumatoid arthritis (RA), pharmacodynamic analyses indicated that complete and sustained CD4 blockade was essential for efficacy (see, Mason, et al. (2002) J. Rheumatol. 29(2):220-229; and Choy, et al. (1996) Arthritis Rheum., 39(1):52-56). A similar degree of CD4 saturation and blockade was needed for the induction of tolerance or hyporesponsiveness to a neoantigen in a preclinical non-human primate model (see Winsor-Hines, et al. (2004) J Immunol., 173(7):4715-4723).

Anti CD4-induced Treg-mediated tolerance induction has been demonstrated in sensitized or previously immunized animal models as well as in the presence of ongoing inflammatory immune responses (e.g., tolerance induction to tissue grafts during active rejection) in murine transplant models. However, tolerance induction in such settings has required higher doses of anti-CD4 antibody, longer treatment duration and/or additional therapeutic agents for efficacy (See, Wise, et al. (1992) Tolerance Induction in the Peripheral Immune System, in Molecular Mechanisms of Immunological Self-Recognition. Cambridge: Academic Press. 149-55 and Marshall, et al. (1996) Transplantation. 62:1614-21).

Nevertheless, in the present study, with multiple doses of antibody, complete saturation of CD4+ sites was only achieved in Cohort 3 (3.0 mg/kg) with no detectable free CD4 sites between doses over the course of the regimen. The antibody dose in Cohort 2 (1.5 mg/kg) was insufficient to achieve complete saturation throughout the thirteen day dosing period. Altering the schedule of TRX1 administration to more frequent dosing would likely achieve sufficient CD4 saturation over the course of the thirteen day regimen to induce durable tolerance foreign antigen in a human albeit insufficient for efficacious treatment of ongoing autoimmune disease. Furthermore, the Treg-mediated non-responsiveness or hyporesponsiveness in previously primed or ongoing immune response settings may require induction of higher ratios of regulatory to effector T cells suggesting that regimens using lower total doses of anti-CD4 antibodies (or CD4-binding fragments or molecules) may be efficacious for human patients without ongoing autoimmune activity. And, addition of therapeutic modalities that reduce on-going inflammation, as well as the implementation of additional rounds of therapy, could also support regimens using lower total doses of anti-CD4 antibodies or CD4-binding fragments or molecules to induce tolerance to or reduce adverse immune response of a human subject to a foreign antigen such as a therapeutic agent.

While not wishing to be bound by any particular mechanistic theory, the increase in the number of antigen-specific Tregs observed would likely be too small to discern against the bulk population of CD4⁺CD25^{hi}Foxp3⁺ T cells. Nevertheless, normal levels of Tregs in circulation do not preclude the possibility of increased numbers at sites of action. In addition to control of B cell responses by Tregs, other mechanisms for maintaining B cell tolerance to self-antigens have been described, and, in the present study, deletion of PhiX-specific B cells, possibly as a consequence of antigen stimulation without sufficient CD4+ T cells help, cannot be ruled out. Indeed, it has been suggested that tolerance may be maintained by a balance of both (see Zheng, et al. (2003) Immunol Rev. 19:675-84).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inducing tolerance or reducing an immune response of a human subject to a foreign antigen, the method comprising:
treating said subject with a regimen, said regimen comprising:
a) one or more administrations of an anti-CD4 antibody or a CD4-binding fragment thereof or a CD4-binding molecule to said subject; and
b) one or more administrations of said foreign antigen to said subject,
wherein during a tolerizing window, the total dose of said antibody administered is 30 mg/kg or less, or the total dose of said fragment administered is the biological equivalent of 30 mg/kg or less, and wherein said tolerizing window is 10 days or less.

2. A method of inducing tolerance or reducing an immune response of a human subject to a foreign antigen, the method comprising:
treating said subject with a regimen, said regimen comprising:
a) one or more administrations of an anti-CD4 antibody or a CD4-binding fragment thereof or CD4-binding molecule to said subject, the first administration comprising at least 0.05 mg/kg but less than 5 mg/kg of said antibody or the biological equivalent of said fragment; and
b) one or more administrations of said foreign antigen to said subject.

3. A method of inducing tolerance or reducing an immune response to a foreign antigen in a human subject, said method comprising:
treating said subject with a regimen, said regimen comprising:
a) at least one administration of an anti-CD4 antibody or a CD4-binding fragment thereof or CD4-binding molecule; and
b) at least one administration of said foreign antigen,
wherein the minimum concentration of said antibody or fragment in the blood of said subject is not less than 5 μg/mL during a tolerizing window.

4. The method of claim 1, wherein the minimum concentration of said antibody or fragment in the blood of said subject is from 5 μg/mL to less than 20 μg/mL during said tolerizing window.

5. The method of claim 1, wherein said tolerizing window is at least three days, at least seven days, at least ten days, or at least fourteen days.

6-10. (canceled)

11. The method of claim 1, wherein said antibody or fragment is administered continuously, and wherein no more than 10 mg/kg of said antibody or the bioequivalent amount of said fragment is administered in the first 24 hour period of said regimen.

12. The method of claim 1, wherein the first administration of said antibody or fragment comprises between 0.5 mg/kg and less than 5.0 mg/kg, between 1.0 mg/kg and 3.0 m mg/kg but less than 5.0 mg/kg, between 50 mg and 350 mg, or between 150 mg and 350 mg of said antibody or the biological equivalent of said fragment.

13-16. (canceled)

17. The method of claim 1, wherein said antibody or fragment or molecule is administered one time during said regimen.

18. (canceled)

19. The method of claim 1, wherein after said tolerizing window, said subject has a reduced immune response to said foreign antigen, wherein the subject is not otherwise immunocompromised.

20. The method of claim 1, wherein said foreign antigen comprises a protein, nucleic acid, or lipid.

21. (canceled)

22. The method of claim 20, wherein said protein comprises an antibody, an enzyme, clotting factor, a cytokine, a hormone, a growth factor, a fusion protein, or a receptor.

23. (canceled)

24. The method of claim 22, wherein said antibody is an anti-CD3 antibody, an anti-tumor necrosis factor (TNF) antibody, an anti-TNF receptor antibody, an anti-CD20 antibody, an anti-glycoprotein IIa/IIb receptor antibody, an anti-IL2-receptor antibody, an anti-epidermal growth factor-receptor antibody, an anti-CD52 antibody, an anti-CD11a antibody, or an anti-HER2 antibody.

25. (canceled)

26. The method of claim 22, wherein the enzyme or clotting factor is factor VIII, factor IX, iduronate-2-sulfatase, alpha-L-iduronidase, alpha-glucosidase, alpha-galactosidase, arylsulfatase B, human deoxyribonuclease, or tissue plasminogen activator.

27. (canceled)

28. The method of claim 22, wherein the cytokine is interferon (IFN)-alpha 2a, IFN-alpha 2b, IFN-beta 1a, IFN-beta 1b, or interleukin-2 (IL-2).

29. (canceled)

30. The method of claim 22, wherein said hormone is animal insulin, recombinant human insulin, recombinant human growth hormone, gonadotropin-releasing hormone, human chorionic gonadotropin, salmon calcitonin, or recombinant human erythropoietin.

31. (canceled)

32. The method of claim 22, wherein said growth factor is granulocyte-macrophage colony stimulating factor (GM-CSF), interleukin 3 (IL-3)-GM-CSF fusion protein, ciliary neurotrophic factor (NTF), or human granulocyte colony stimulating factor (G-CSF).

33. (canceled)

34. (canceled)

35. The method of claim 22, wherein said receptor comprises TNF receptor.

36. The method of claim 20, wherein said nucleic acid comprises a gene therapy delivery vehicle.

37. The method of claim 1, wherein said antibody or fragment or molecule is administered every other day during said tolerizing window.

38. The method of claim 1, wherein said administration of said antibody or fragment or molecule comprises first and second administrations of said antibody or fragment or molecule, wherein said second administration is between five and eight days after said first administration.

39. (canceled)

40. The method of claim 1, said method further comprising at least one follow-up regimen, said follow-up regimen comprising at least one administration of said antibody or fragment or molecule to said subject.

41. (canceled)

42. (canceled)

43. The method of claim 1, further comprising administering at least one compound selected from the group consisting of an antihistamine, an antiemetic, an immunosuppressant, or an anti-inflammatory.

44. The method of claim 1, wherein the antibody or fragment has been modified to reduce binding to one or more Fc (gamma) receptors compared to the corresponding antibody or fragment without the modification.

45. The method of claim 1, wherein the antibody is a monoclonal antibody, a humanized antibody, and/or non-depleting.

46. (canceled)

47. (canceled)

48. The method of claim 1, wherein the antibody or fragment has the six complementarity determining regions (CDRs) of TRX1.

49. The method of claim 1, wherein the antibody is aglycosylated.

50. The method of claim 1, wherein the antibody is designated TRX1 and contains either a leucine residue or a proline residue at position 117.

51. (canceled)

52. (canceled)

53. The method of claim 45, wherein the antibody or fragment has a further modification that reduces serum clearance of the further modified antibody by at least 38% as compared to the corresponding antibody or fragment without the further modification or increases binding of the antibody or fragment to FcRn compared to the binding of the corresponding antibody or fragment without the further modification.

54. (canceled)

55. The method of claim 53, wherein the antibody is mMTRX1011A.

56. (canceled)

57. (canceled)


SEQUENCE LISTING (4 sequences)

SEQ ID NO: 1 - protein, 237 aa, Artificial Sequence
  a light chain of humanized antibody TRX1
METDTILLWVLLLWVPGSTGDIVMTQSPDSLAVSLGERATINCKASQSVDYDGDSYMNWYQQKPGQPPKLLIYVASNLESGVPQRFSGSGSGTDFTLTISSLQAEDVAVYYCQQSLQDPPTFGGGTKVEIKRTVAALSVFIFPPSDEQLKSGTASVVCLLNNFYPREAKVQWKVDNALQSGNSQESVTEQDSKDSTSLSSTLTLSKADYEKHKVYACEVTHQGLSSPVTKSFNRGEC

SEQ ID NO: 2 - DNA, 717 bp, Artificial Sequence
  a light chain from a TRX1 humanized antibody
atggagacagacacaatcctgctatgggtgctgctgctctgggttccaggctccactggtgacattgtgatgacccaatctccagattctttggctgtgtctctaggtgagagggccaccatcaactgcaaggccagccaaagtgttgattatgatggtgatagttatagtaactggtatcaacagaaaccaggacagccacccaaactcctcatctatgttgcatccaatctagagtctggggtcccagacaggtttagtggcagtgggtctgggacagacttcaccctcaccatcagttctctgcaggcggaggatgttgcagtctattactgtcagcaaagtcttcaggaccctccgacgttcggtggaggtaccaaggtggaaatcaaacgaactgtggctgcactatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - DNA, 1404 bp, Artificial Sequence
  a heavy chain of humanized antibody TRX1
atggaatggatctggatctttctcctcatcctgtcaggaactcgaggtgtccagtcccaggttcagctggtgcagtctggagctgaagtgaagaagcctggggcttcagtgaaggtgtcctgtaaggcttctggatacacattcactgcctatgttataagctgggtgaggcaggcacctggacagggccttgagtggatgggagagatttatcctggaagcggtagtagttattataatgagaagttcaagggcagggtcacaatgactagagacacatccaccagcacagtctacatggaactcagcagcctgaggtctgaggacactgcggtctattactgtgcaagatccggggacggcagtcggtttgtttactggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 467 aa, Artificial Sequence
  a heavy chain from a TRX1 humanized antibody
MEWIWIFLLILSGTRGVQSQVQLVQSGAEVKKPGASVKVSCKASGYTFTAYVISWVRQAPGQGLEWMGEIYPGSGSSYYNEKFKGRVTNTRDTSTSTVYMELSSLRSEDTAVYYCARSGDGSRFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLQSDGSFFLYSKLTVDKSRWQQGNVFSCSVMHEALHNHYTQKSLSLSPGK